(19) United States
(12) Patent Application Publication
(10) Pub. No.: US 2022/0186202 A1
(43) Pub. Date: Jun. 16, 2022

(54) CARBONIC ANHYDRASE VARIANTS FOR IMPROVED CO2 CAPTURE

(71) Applicant: SAIPEM S.P.A., San Donato Milanese (Milano) (IT)

(72) Inventors: Richard DAIGLE, San Donato Milanese (Milano) (IT); Mikael BEDARD, San Donato Milanese (Milano) (IT); Eric MADORE, San Donato Milanese (Milano) (IT); Sylvie FRADETTE, San Donato Milanese (Milano) (IT); Normand VOYER, San Donato Milanese (Milano) (IT)

(21) Appl. No.: 17/598,119

(22) PCT Filed: Mar. 17, 2020
(86) PCT No.: PCT/IB2020/052410

§ 371 Date: Sep. 24, 2021

Related U.S. Application Data
(60) Provisional application No. 62/823,744, filed on Mar. 26, 2019.

(51) Int. Cl. C12N 9/88

(52) U.S. Cl. CPC C12N 9/88; C12Y 402/01001

(57) ABSTRACT

Recombinant carbonic anhydrase variants having improved solubility and/or thermostability for enzyme-enhanced CO2 capture are provided. Host cells, methods, and processes relating to same are also provided.

The present description relates to enzyme-enhanced processes for capturing CO₂ from a CO₂-containing effluent or gas. More particularly, described herein are recombinant carbonic anhydrase variants having improved solubility and/or thermostability under conditions relevant to carbonic anhydrase-based CO₂ capture processes.

### BACKGROUND

Increasingly dire warnings of the dangers of climate change by the world's scientific community combined with greater public awareness has prompted increased momentum towards reducing man-made greenhouse gas (GHGs) emissions, most notably carbon dioxide. Fossil fuel-burning power plants represent one of the largest sources of CO₂ emissions worldwide and thus implementation of an effective GHG reduction system will require mitigation of CO₂ emissions generated by this sector. Carbonic anhydrase-enhanced CO₂ capture processes provide one of the most promising carbon capture, utilization and storage solutions, however there are several challenges related to its widespread commercial implementation. One of the principal challenges is improving economic feasibility. The main operating cost associated with carbonic anhydrase-enhanced CO₂ capture processes is replenishing depleted or inactive carbonic anhydrase enzyme. There is thus a need for improved carbonic anhydrases that can address at least some of these challenges.

### SUMMARY

Recombinant carbonic anhydrase variants having improved solubility and/or thermostability for enzyme-enhanced CO₂ capture are described herein. While the use of carbonic anhydrase enzymes and variants thereof having enhanced thermostability can dramatically reduce operating costs, some enzymes and variants exhibiting improved thermostability are associated with an undesired concomitant decrease in enzyme solubility, which may preclude their implementation in real-world CO₂ capture operations. For instance, Example 1 shows that thermostable wild-type Thermovibrio ammonificans carbonic anhydrase (TACA) may be prone to aggregation/precipitation when subjected to elevated temperatures (e.g., 80° C.) in alkaline carbonate solutions.

Interestingly, a single amino acid substitution, R156E, increased the enzyme's solubility approximately two-fold at 80° C. in an alkaline carbonate solution (see Table 1). This single amino acid substitution resulted in a decrease in the calculated isoelectric point (pI) of the enzyme from 8.8 to 8.3. A combination of random mutagenesis and rational design approaches, followed by empirical testing, were thus employed to engineer and express TACA variants retaining carbonic anhydrase activity yet having progressively lower isoelectric points, for example ranging from 8.3 to 5.9. The TACA variants having lower pI values generally exhibited higher solubility in alkaline carbonate solutions (Table 1).

With the goal of finding novel mutations having a beneficial impact on thermostability and/or solubility, large-scale random mutagenesis screening was performed starting from different templates encoding functional TACA variants engineered to have progressively lower isoelectric points (Examples 2 and 3). To simplify comparison of different individual TACA variants, as well as their impact on their respective templates, the results of extensive solubility and thermostability testing were converted to “solubility scores” and “stability scores”. Because both solubility and thermostability were found to be often interrelated in terms of their benefit in CO₂ capture processes, “overall scores” combining both solubility and stability scores were also calculated for each variant, which enabled the different variants to be ranked in terms of their potential attractiveness for implementation in CO₂ capture processes.

Some beneficial amino acid substitutions were found to improve both thermostability and solubility, while other beneficial substitutions were found to improve either thermostability or solubility. Interestingly, it was found that improving the solubility of an enzyme often reduced the effective concentration of that enzyme required to achieve a given CO₂ capture efficiency, as compared to an enzyme having the same thermostability albeit with lower solubility. Furthermore, it was generally found that individual amino acid substitutions that had a beneficial effect in terms of solubility and/or thermostability on their parent templates, also had beneficial effects when introduced in different templates. Moreover, it was found that combining multiple individual variants having beneficial effects on solubility and/or thermostability on the same template resulted in recombinant carbonic anhydrase polypeptides that generally outperformed enzymes having only the corresponding single variants.

In some aspects, described herein are recombinant carbonic anhydrase polypeptides having carbonic anhydrase activity comprising an amino acid sequence having at least 60% identity with SEQ ID NO: 5 and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223, wherein said recombinant carbonic anhydrase polypeptide has increased solubility and/or increased thermostability as compared to a corresponding carbonic anhydrase polypeptide lacking said one or more amino acid differences.

In some aspects, described herein are recombinant carbonic anhydrase polypeptides having carbonic anhydrase activity comprising an amino acid sequence having at least 60% identity with SEQ ID NO: 5, wherein said recombinant carbonic anhydrase polypeptide comprises the residue(s): 3E; 6R; 9A or 9N; 11L, 11P, or 11Y; 15L; 17Y; 18I, 18L, 18R, or 18S; 20K or 20L; 24I, 24M, or 24V; 25F; 27R; 38D, 38R, or 38T; 39H, 39I, 39L, 39R, or 39W; 48L, 48Q, or 48T; 51D, 51E, 51F, 51M, or 51P; 64T; 73E or 73L; 77F; 79E, 79L or 79W; 88E, 88I, 88L, 88R, 88T, or 88V; 105F; 116E or 116R, 119D or 119M; 128E, 128K, 128R, or 128T; 130A, 130D, 130E, 130F, 130H, 130K, 130Q, 130R, 130S, 130T, 130V, 130W, or 130Y; 137D or 137E; 138E or 138L; 145D or 145E; 148F, 148V, or 148W; 149I; 154D, 154K, 154P, or 154V; 156V; 158Y; 160D or 160Q; 166E or 166V; 167L; 168E, 168F, 168R, or 168W; 170F; 192D; 195D or 195E; 199A, 199D, or 199K; 203R or 203V; 206R; 210H; 216T; 219I; 223I, 223L, or 223V; 226R; or any combination thereof.

In some aspects, described herein are recombinant carbonic anhydrase polypeptides having carbonic anhydrase activity comprising an amino acid sequence having at least 60% identity with SEQ ID NO: 5, wherein said recombinant carbonic anhydrase polypeptide is engineered to have an isoelectric point (pI) below that of SEQ ID NO: 2, 3 or 4, and has a solubility greater than that of SEQ ID NO: 2, 3 or 4 after 24 hours at 80° C. in an alkaline carbonate solution, such as a solution ranging from 1.38 to 1.85 M K₂CO₃ with alpha varying from 0.60 to 0.89.

In some aspects, described herein are isolated polynucleotides encoding the above mentioned. recombinant carbonic anhydrase polypeptides.

In some aspects, described herein are expression or cloning vectors comprising the above mentioned isolated polynucleotides.

In some aspects, described herein are host cells comprising the above mentioned isolated polynucleotide, or the above mentioned expression vectors.

In some aspects, described herein are method of producing a recombinant carbonic anhydrase polypeptide, the method comprising culturing the above mentioned host cell under conditions enabling the expression of the above mentioned recombinant, carbonic anhydrase polypeptides, and recovering the recombinant carbonic anhydrase polypeptide.

In some aspects, described herein is the use of the above mentioned recombinant carbonic anhydrase polypeptides in an industrial process for capturing CO₂ from a CO₂-containing effluent or gas.

In some aspects, described herein are processes for absorbing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptide defined herein to catalyze the hydration reaction of the dissolved CO₂ into bicarbonate and hydrogen ions or the reverse reaction.

In some aspects, described herein is a stock or feed solution comprising the recombinant carbonic anhydrase polypeptide as defined herein at a concentration of at least 5, 6, 7, 8, 9, 10, 11, or 12 g/L.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method step.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows an amino acid sequence alignment between SEQ ID NO: 1, SEQ ID NO: 2, SEQ ID NO: 3, and SEQ ID NO: 4.

FIG. 2 shows stability scores of some variants of SEQ ID NO: 3 (FIG. 2A) and of some variants of SEQ ID NO: 4 (FIG. 2B)

FIG. 3 shows absorbance at 595 nm of different K₂CO₃ solutions containing various carbonic anhydrase (CA) enzymes (FIG. 3A to FIG. 3L) at a concentration of 2 g/L after a 24 h-incubation at 30° C. (FIGS. 3A, 3C, 3E, 3G, 3I, and 3K), and 70° C. (FIGS. 3B, 3D, 3F, 3H, 3J, and 3L). The K₂CO₃ concentration ranges from 1.38 M to 1.85 M. CO₂ loading of the solutions varied from 0.60 to 0.89 mol C/mol K⁺. Because of KHCO₃ solubility limits, solutions with a CO₂ loading of 0.89 mol C/mol K⁺ are restricted to solutions having a K₂CO₃ concentration ranging from 1.38 M to 1.45 M. Similarly, solutions having a CO₂ loading of 0.84 mol C/mol K⁺ are restricted to solutions having a K₂CO₃ concentration ranging from 1.38 M to 1.65 M inclusively. The absorbance at 595 nm is related to the amount of insoluble/aggregated enzyme in solution.

FIG. 4 shows half-life gains (%) of various CA enzymes over the half-life of SEQ ID NO: 4 in 1.45 M K₂CO₃ alpha 0.70 mol C/mol K⁺ at 70, 85 and 95° C.

FIG. 5 shows absorbance at 595 nm of various K₂CO₃ solutions containing CA enzymes derived from SEQ ID NO: 4 at a concentration of 2 g/L after a 24 h-incubation at 30° C. (FIGS. 5A, 5C, 5E, 5G, and 5I) and 70° C. (FIGS. 5B, 5D, 5F, 5H and 5J). The K₂CO₃ concentration of the solutions ranges from 1.38 M to 1.85 M. CO₂ loading ranges from 0.60 to 0.89 mol C/mol K⁺. Because of KHCO₃ solubility limits, solutions with a CO₂ loading of 0.89 mol C/mol K⁺ are restricted to solutions having a K₂CO₃ concentration ranging from 1.38 M to 1.45 M. Similarly, solutions having a CO₂ loading of 0.84 mol C/mol K⁺ are restricted to solutions having a K₂CO₃ concentration ranging from 1.38 M to 1.65 M inclusively. The absorbance at 595 nm is related to the amount of insoluble/aggregated enzyme in solution.

FIG. 6 shows an example of a multiple sequence alignment of the carbonic anhydrases of SEQ ID NOs: 1 and 12-20, originating from different organisms.

FIG. 7 shows a phylogenic tree analysis corresponding to the multiple sequence alignment shown FIG. 6.

### SEQUENCE LISTING

This application contains a Sequence Listing in computer readable form created Mar. 24, 2019 having a size of about 44 KB. The computer readable form is incorporated herein by reference.

| SEQ ID NO: | Description |
|---|---|
| 1 | Wild-type TACA sequence with N terminus modified for improved bacterial expression; |
| | pI of 8.8 |
| 2 | TACA variant having pI of 8.3 |
| | (SEQ ID NO: 1 + R156E) |
| 3 | TACA variant having pI of 7.2 |
| | (SEQ ID NO: 1 + K27R, N38D, K88R, K116R, N119D, K128R, R156E, E160D, D168E, |
| | E192D, E199D, K203R, K206R, V216T, L219I) |
| 4 | TACA variant having pI of 6.1 |
| | (SEQ ID NO: 1 + Y77F, V79E, K88E, Y105F, K116E, K128E, E137D, E145D, R156E, D168E, |
| | Y170F, E195D, E199D, V216T, L219I, K226R) |
| 5 | Amalgam of SEQ ID NOs: 2-4 |
| 6 | TACA variant having pl of 6.1 |
| | (SEQ ID NO: 4 + S39I, E128K, T154D, K223I) |
| 7 | TACA variant having pl of 6.1 |
| | SEQ ID NO: 4 + S39I, G130A, T154D, K223I) |
| 8 | TACA variant having pl of 5.8 |
| | (SEQ ID NO: 4 + S39I, G130A, T154D, K223L) |
| 9 | TACA variant having pI of 5.9 |
| | (SEQ ID NO: 4 + S39I, G130A, T154P, D195E, K223I) |
| 10 | TACA variant having pI of 5.9 |
| | (SEQ ID NO: 4 + S39I, G130A, T154P, D195E, K223L) |
| 11 | TACA variant having pI of 5.9 |
| | (SEQ ID NO: 4 + S39I, K223L) |
| 12 | Carbonic anhydrase from Persephonella marina (WP_015898908.1) |
| 13 | Carbonic anhvdrase from Persephonella sp. KM09-Lau-8 (WP_029522463.1) |
| 14 | Carbonic anhydrase from Persephonella sp. IF05-L8 (WP_029521561.1) |
| 15 | Carbonic anhydrase from uncultured bacterium (AVN84966.1) |
| 16 | Carbonic anhydrase from Persephonella hydrogeniphila (WP_096999253.1) |
| 17 | Carbonic anhydrase from Aquificae bacterium (RMD45622.1) |
| 18 | Carbonic anhydrase from Caminibacter mediatlanticus (WP_007474387.1) |
| 19 | Carbonic anhydrase from Hydrogenimonas sp. (BBG65557.1) |
| 20 | Carbonic anhydrase from Hydrogenimonas sp. (RUM45284.1) |

### DETAILED DESCRIPTION

The present description relates to recombinant carbonic anhydrase variants having improved solubility and/or thermostability for enzyme-enhanced CO₂ capture, as well as polynucleotides, vectors, host cells, methods, and processes relating to same.

Industrial carbonic anhydrase-based CO₂ capture operations generally involve exposing the enzyme to repeated temperature fluctuations that may range from 10° C. to 98° C., depending on the particular process conditions employed. PCT patent application WO/2016/029316 describes methods for enzyme-enhanced CO₂ capture utilizing Thermovibrio ammonificans carbonic anhydrase (TACA), or functional derivatives thereof, for catalyzing the hydration reaction of CO₂ into bicarbonate and hydrogen ions and/or catalyzing the desorption reaction to produce CO₂ gas. PCT patent application WO/2017/035667 describes variants of TACA engineered for improved performance in CO₂-capture operations, notably TACA variants having improved thermostability in the context of an alkaline carbonate absorption solution as compared to the wild type enzyme.

While the use of carbonic anhydrase enzymes and variants having enhanced thermostability can dramatically reduce operating costs for example by increasing enzyme half-life, some enzymes and variants exhibiting improved thermostability are associated with an undesired concomitant decrease in enzyme solubility, which may preclude their implementation in real world CO₂ capture operations. For instance, Example 1 shows that thermostable wild-type Thermovibrio ammonificans carbonic anhydrase (TACA) may be prone to aggregation/precipitation when subjected to elevated temperatures (e.g., 80° C.) in alkaline carbonate solutions.

Ideally, an enzyme deployed in a commercial-scale CO₂ capture operation must remain in solution in an active form (e.g., aggregate- and/or precipitate-free) throughout the CO₂ capture process conditions, because even incremental precipitation/aggregation of the enzyme at any point during a CO₂ absorption/desorption thermal cycle would lower the effective concentration of the enzyme in solution over time, thereby requiring fresh enzyme to be added more frequently. Conversely, an enzyme having improved solubility and/or enhanced resistance to aggregation throughout the CO₂ capture process conditions may present additional technical and practical advantages such as: potentially exhibiting greater stability at the gas-liquid interphase (by reducing the affinity for the interface which is hydrophobic); facilitating solubilization of dried or lyophilized enzyme; minimizing enzyme loss due to aggregation (enzymatically inactive soluble aggregates) and/or precipitation (insoluble aggregates); offering the possibility of preparing highly concentrated “feed” solutions for use in CO₂ capture processes; enabling a more concentrated stock solution to be shipped from enzyme suppliers, thereby reducing shipping costs.

Interestingly, a single amino acid substitution, R156E, was found to increase the solubility of TACA approximately two-fold at 80° C. in an alkaline carbonate solution (see Table 1). This single amino acid substitution resulted in a slight decrease in the calculated isoelectric point (pI) the enzyme from 8.8 to 8.3. A combination of random mutagenesis and rational design approaches, followed by empirical testing, were thus employed to engineer and express TACA variants retaining carbonic anhydrase activity yet having progressively lower isoelectric points ranging from 8.3 to 5.9. The TACA variants tested having lower pI values generally exhibited improved solubility in alkaline carbonate solutions (Table 1).

With the goal of finding novel mutations having a beneficial impact on thermostability without negatively impacting solubility, three TACA variants having enzymatic activity but different isoelectric points were employed as starting point templates for random mutagenesis screening, as described in Examples 2 and 3. The templates used for the random mutagenesis screening are represented by SEQ ID NO: 5, which is an amalgam of SEQ ID NOs: 2, 3 and 4 (see Table 1). To simplify comparison of different individual TACA variants identified, as well as their impact on their respective starting point templates, the data from solubility and thermostability testing were converted to “solubility scores” and “stability scores”. Because both solubility and thermostability were found to be often interrelated in terms of their benefit in CO₂ capture processes, “overall scores” combining both solubility and stability scores were also calculated for each variant, which enabled the different variants to be ranked in terms of their potential suitability fix implementation in CO₂ capture processes.

Accordingly, described herein are amino acid substitutions shown to have a beneficial impact, individually and/or collectively, on the solubility and/or thermostability of carbonic anhydrase enzymes derived from wild-type TACA (represented herein by SEQ ID NO: 1). For greater clarity, the expression “wild-type TACA” as used herein is intended to refer to the amino acid sequence of SEQ ID NO: 1, which generally corresponds to the amino acid sequence of naturally occurring TACA (e.g., Accession No. WP_013538320.1), except that the N-terminal part of the enzyme is optimized as described in WO/2017/035667 for increased enzyme production in a bacterial expression system. Some beneficial amino acid substitutions described herein were found to improve both thermostability and solubility, while other beneficial amino acid substitutions were found to improve either thermostability or solubility. Interestingly, it was found that improving the solubility of an enzyme often reduced the effective concentration of that enzyme required to achieve a given CO₂ capture efficiency, as compared to an enzyme having the same thermostability albeit with lower solubility. As used herein, the expression “effective enzyme concentration” refers to a concentration of enzyme that causes a defined magnitude of response in a given system, wherein the enzyme concentration incudes all forms of the enzyme, such as soluble enzyme, insoluble enzyme, and soluble aggregates of the enzyme. Furthermore, it was generally found that individual amino acid substitutions that had a beneficial effect in terms of solubility and/or thermostability on their parent templates, also had beneficial effects when introduced in different templates. Moreover, it was found that combining multiple individual variants having beneficial effects on solubility and/or stability on the same template resulted in recombinant carbonic anhydrase enzymes that generally outperformed enzymes having only the corresponding single variants.

In some aspects, described herein are recombinant carbonic anhydrase polypeptides having carbonic anhydrase activity comprising an amino acid sequence having at least 60% identity with any one of SEQ NOs: 1 to 5, and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223. Amino acid substitutions at these positions are shown herein to have a beneficial impact on enzyme solubility and/or thermostability (e.g., in an alkaline carbonate solution as described herein), as compared to corresponding carbonic anhydrase polypeptides lacking the amino acid substitutions.

As used herein, the expression “alkaline carbonate solution” generally refers to a solution containing a carbonate compound or carbonate ions having an alkaline pH (e.g., pH of greater than 7 at room temperature) that is suitable for evaluating the improved thermostability and/or solubility of TACA enzymes and variants described herein. For example, in some embodiments, the alkaline carbonate solution may have a carbonate concentration of 0.1 to 3 M, 0.5 to 2 M, 1 to 2 M, or 1.25 to 1.75 M. In particular embodiments, the alkaline carbonate solution may be a solution ranging from 1.38 to 1.85 M carbonate (e.g., K₂CO₃) with alpha varying from 0.60 to 0.89, such as described in the titration solubility testing shown in Example 3.

As used herein, the term “alpha” in the context of alkaline carbonate solutions refers to the CO₂ loading and corresponds to the ratio of the concentration of carbon to potassium in the solution (i.e., CO₂ loading or alpha=[Carbon]/[Potassium]). For example, a pure solution of 1.45 M K₂CO₃ has an alpha of [1.45]/[2×1.45]=0.5, while a pure solution of 2.9 M KHCO₃ has an alpha of [2.9]/[2.9]=1. A mixture of 0.87 M K₂CO₃+1.16 M KHCO₃ has an alpha of [0.87+1.16]/[(0.87×2)+1.16]=2.03/2.9=0.7.

As used herein, the expression “recombinant carbonic anhydrase polypeptide(s)” refers to non-naturally occurring enzymes capable of catalyzing the hydration of carbon dioxide engineered or produced using recombinant technology. In some embodiments, the recombinant carbonic anhydrase polypeptides described herein may comprise any type of modification (e.g., chemical or post-translational modifications such as acetylation, phosphorylation, glycosylation, sulfatation, sumoylation, prenylation, ubiquitination, etc.). For further clarity, polypeptide modifications are envisaged so long as the modification does not destroy the carbonic anhydrase activity of the carbonic anhydrase polypeptides described herein. Methods for measuring carbonic anhydrase activity are described for example in WO/2016/029316 and/or WO/2017/035667.

In some embodiments, the recombinant carbonic. anhydrase polypeptides described herein may comprise the residue(s): 3E; 6R; 9A or 9N; 11L, 11P, or 11Y, 15L; 17Y; 18I, 18L, 18R, or 18S; 20K or 20L; 24I, 24M, or 24V; 25F; 27R; 38D, 38R, or 38T; 39H, 39I, 39L, 39R, or 39W; 48L, 48Q, or 48T; 51D, 51E, 51E, 51M, or 51P; 64T; 73F, or 73L; 77F; 79E, 79L or 79W; 88E, 88I, 88L, 88R, 88T, or 88V; 105F; 116E or 116R, 119D or 119M, 128E, 128K, 128R, or 128T; 130A, 130D, 130E, 130F, 130H, 130K, 130Q, 130R, 130S, 130T, 130V, 130W, or 130Y; 137D or 137E; 138E or 138L; 145D or 145E; 148F, 148V, or 148W; 149I; 154D, 154K, 154P, or 154V; 156V, 158Y; 160D or 160Q; 166E or 166V; 167L; 168E, 168F, 168R, or 168W; 170F; 192D; 195D or 195E; 199A, 199D, or 199K, 2038 or 203V; 206R; 210H; 216T; 219I; 223I, 223L, or 223V; 226R; or any combination thereof. These amino acid substitutions are ones that are either shown experimentally herein to be associated with a solubility score, stability score, or an overall score of greater than 1.0, indicating their presence had a beneficial impact on enzyme solubility and/or thermostability in an alkaline carbonate solution, or were found on the template carbonic anhydrases of SEQ ID NOs: 3 and 4 having increased solubility as compared to wild-type TACA at 80° C. in alkaline carbonate solution.

In some embodiments, the recombinant carbonic anhydrase polypeptides described herein may comprise at least two, three, tour, five, six, seven, eight, nine, ten, eleven, twelve, thirteen, fourteen, fifteen, sixteen, seventeen, eighteen, nineteen, or twenty of the residues defined above. In some embodiment, all combinations of the beneficial amino acid substitutions are described herein.

In some embodiments, the recombinant carbonic anhydrase polypeptides described herein may comprise the residue(s): 3E; 11P; 18S; 24I; 38D; 39I, 39L, 39H,39R, 39L, or 39I; 88I; 130S or 130D; 154D or 154P; 223L or 223I; 130S and 154D; 130A and 154D; 130D and 154D; 130A, 154P, and 195E; 15L, 38D, and 128K; 195E and 223I; 25F, 38D, and 128K; 38D and 128K; 38D, 128K and 137E; 38D, 128K, 137E, and 154D; 38D, 128K, 137E, and 154P; 38D, 128K, and 145E; 38D, 128K, and 148W; 38D, 128K, and 160Q; 38D, 128K, and 167L; 38D, 128K; and 168D; 38D, 128K, and 195E; 38D, 128K, and 199A; 38D, 128K, and 216V; 38D, 128K, and 219L; 38D, 128K, and 226K; 38D, E88I, and 128K; 38D, E88K, and 128K; 539I, 128K, 154D, and 223I; 539I, 130A, 154P, 195E, and 223I; 39I, 130A, 154P, 195E, and 223L; 39I, 130A, 154D, and 223L; 539I, 130A, 154D, and 223I; 39I and 195E; 39I and 223I; 39L and 223L; 39L and 223I; 39I and 223L. These amino acid substitutions are ones that are either shown experimentally herein to be associated with overall scores of greater than 2.0, indicating their presence had a beneficial impact on enzyme solubility and/or thermostability in an alkaline carbonate solution.

In some embodiments, the recombinant carbonic anhydrase polypeptides described herein are engineered to have lower isoelectric points, as compared to wild-type or parent enzymes lacking the engineering, As used herein, “isoelectric point” or “pI” refers to the pH at which a polypeptide carries no net electrical charge or is electrically neutral, which can be determined experimentally or theoretically (calculated). In some embodiments, the pI of a polypeptide described herein may be determined experimentally by methods known in the art, such as isoelectric focusing. In other embodiments, the pI of a polypeptide described herein may be a theoretical pI calculated using an algorithm, for example, based on the use of the Henderson-Hasselbalch equation with different pK values, In some embodiments, the pI of a polypeptide described herein may be computed using an available online tool, such as the Compute pI/Mw online tool available at the ExPASy Bioinformatics Resource Portal (https://web.expasy.org).

It is shown herein in Example 1 that engineering wild-type TACA to lower its pI may help increase the solubility of the enzyme, particularly at elevated temperatures (e.g., 80° C.) in alkaline carbonate solution. Indeed, Table 1 shows that the carbonic anhydrase variants of SEQ ID NOs: 2-7 having progressively lower pIs (i.e., from 8.3 to 6.9) are associated with progressively higher solubilities at 80° C. in alkaline carbonate solution (e.g., 1.45 M K₂CO₃ alpha 0.7), as compared to wild-type TACA (SEQ ID NO: 1) having a pI of 8.8. Accordingly, in some embodiments, the recombinant carbonic anhydrase polypeptides described herein may be engineered to have an isoelectric point (pI) below that of SEQ ID NO: 2, 3, or 4. In some embodiments, the recombinant carbonic anhydrase polypeptides described herein may have a pI at or below 8.3, 8.2. 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, or 4.5. In some embodiments, the recombinant carbonic anhydrase polypeptides described herein may have a pI of 4 to 8, 4.5 to 7.5, 5 to 7, 5.5 to 6.5, or 5 to 6.

In some embodiments, the recombinant carbonic anhydrase polypeptides described herein comprising one or more amino acid differences as compared to SEQ ID NO: 1 at residue positions described herein may exhibit increased solubility and/or increased thermostability as compared to a corresponding parent carbonic anhydrase polypeptide lacking the one or more amino acid differences (herein referred to as “control carbonic anhydrase polypeptide”), particularly in an alkaline carbonate solution. In some embodiments, solubility and/or thermostability testing may be performed as described in Examples 1-3. In some embodiments, thermostability testing may be performed as described for example in WO/2017/035667.

In some embodiments, the recombinant carbonic anhydrase polypeptides described herein may exhibit increased solubility after a 24-hour exposure at 22° C. or 70° C. in alkaline carbonate solution, as compared to a control carbonic anhydrase polypeptide. In some embodiments, the recombinant carbonic anhydrase polypeptides described herein may exhibit increased solubility after a 24-hour exposure at 80° C. in an alkaline carbonate solution. In some embodiments, the recombinant carbonic anhydrase polypeptides described herein may exhibit increased solubility, as determined by titration solubility testing. In some embodiments, the titration solubility testing may be performed by measuring turbidity of 2 g/L of the recombinant carbonic anhydrase polypeptide in solutions ranging from 1.38 to 1.85 M K₂CO₃ with alpha varying from 0.60 to 0.89, as described in Example 3. In particular embodiments, the recombinant carbonic anhydrase polypeptides described herein may have a solubility of greater than 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12 g/L after 24 hours at 80° C. in an alkaline carbonate solution.

In some embodiments, the recombinant carbonic anhydrase polypeptides described herein may exhibit increased thermostability as compared to a control carbonic anhydrase polypeptide, after a 72-hour exposure at 85° C. in an alkaline carbonate solution. In some embodiments, the recombinant carbonic anhydrase polypeptides described herein may exhibit increased thermostability as compared to a control carbonic anhydrase polypeptide, after a 16-hour exposure at 95° C. in an alkaline carbonate solution.

In some embodiments, the recombinant carbonic anhydrase polypeptides described herein may comprise an amino acid sequence having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96,%, 97%, 98%, or 99% identity with any one of the SEQ ID NOs described herein relating to Thermovibrio ammonificans carbonic anhydrase (e.g., SEQ ID NOs: 1-11).

Techniques for determining amino acid “sequence identity” are known in the art. For example, there is the widely used program Emboss Needle (https://www.ebi.ac.uk) exploiting the Needleman-Wunsch algorithm. This program aligns optimally two sequences given as input according to chosen similarity matrix (e.g., BLOSOM62) and other parameters (e.g., gap opening, gap extent). As output, it returns a sequence alignment, the number of gap(s) that it includes, as well a similarity and identity percentages. The identity percentage is calculated by dividing the total number of residues for which the same amino acid is found in both sequences by the sequence length of the reference enzyme (e.g., SEQ ID NO: 4 having 226 residues). For greater clarity, when calculating the percentage identity of a given sequence relative to a reference sequence that defines more than a single amino acid possibility at a given residue position (e.g., SEQ ID NO: 5), the given sequence is considered as a match to the reference sequence at that residue position if the given sequence contains any one of the possible amino acids defined for that position by the reference sequence.

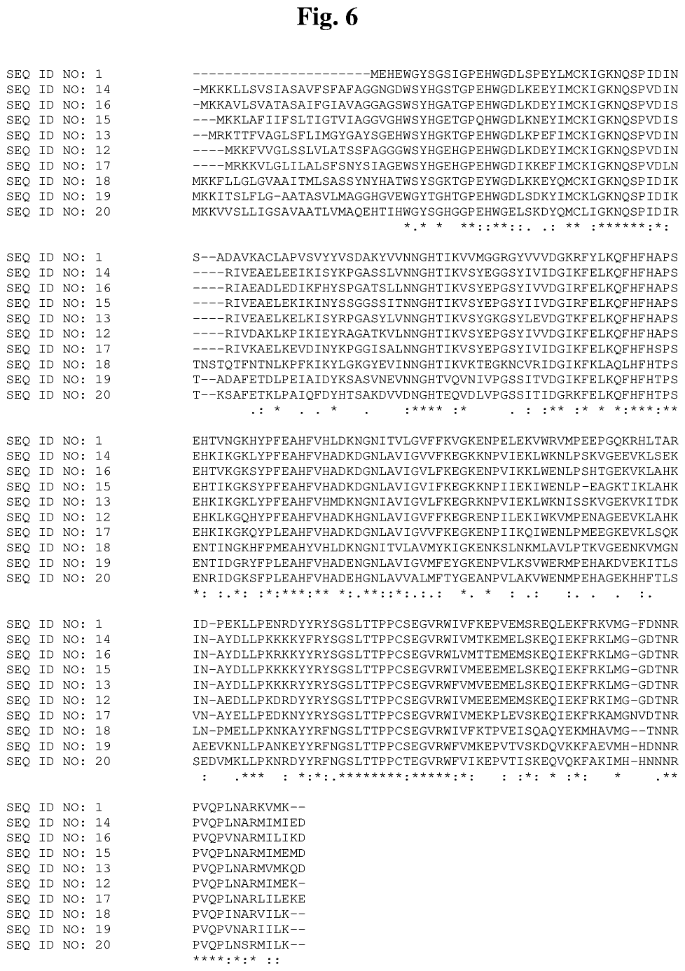

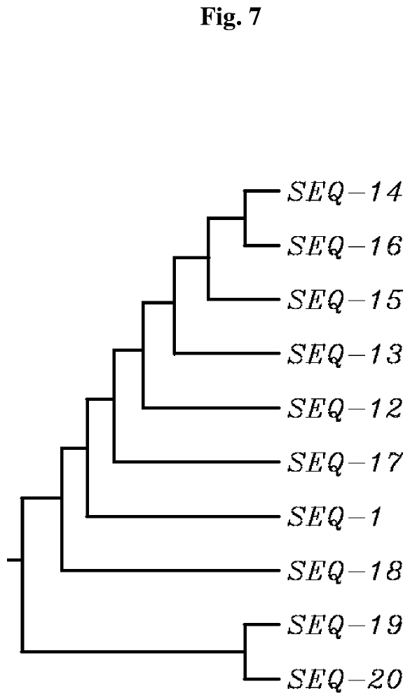

In some embodiments, the recombinant carbonic anhydrase polypeptides described herein may comprise an amino acid sequence having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with a wild-type carbonic anhydrase from Persephonella marina (Accession No: WP_015898908.1, SEQ ID NO: 12), Persephonella sp. KM09-Lau-8 (WP_029522463.1; SEQ ID NO: 13), Persephonella sp. IF05-L8 (WP_029521561.1; SEQ ID NO: 14), uncultured bacterium (AVN84966.1; SEQ ID NO: 15), Persephonella hydrogeniphila (WP_096999253.1; SEQ ID NO: 16), Aquificae bacterium (RMD45622.1; SEQ ID NO: 17), Caminibacter mediatlanticus (WP_007474387.1; SEQ ID NO: 18), Hydrogenimonas sp. (BBG65557.1; SEQ ID NO: 19), or Hydrogenimonas sp. (RUM45284.1; SEQ ID NO: 20). The foregoing carbonic anhydrases represent some of the closest orthologs to TACA in terms of amino acid sequence conservation, and are also from thermophilic organisms. In some embodiments, the amino acid substitutions demonstrated herein to impart a beneficial effect in terms of solubility and/or thermostability to different TACA templates may be engineered into the corresponding residue positions in the background of any one of SEQ NOs: 12-20. Corresponding residue positions may be identified by persons of skill in the art by performing multiple sequence alignments with the TACA sequences described herein, as shown in FIG. 6.

In some aspects, described herein are isolated polynucleotides encoding the recombinant carbonic anhydrase polypeptides as defined herein. In some embodiments, the isolated polynucleotide may be operably linked to a heterologous promoter.

In some aspects, described herein are expression or cloning vectors comprising the isolated polynucleotides as defined herein.

In some aspects, described herein are host cells comprising the isolated polynucleotides as defined herein, or the expression or cloning vectors as defined herein. In some embodiments, the host cells may be bacterial cells, yeast cells, or fungal cells.

In some aspects, described herein are methods of producing recombinant carbonic anhydrase polypeptides, the method comprising culturing the host cells as defined herein under conditions enabling the expression of the recombinant carbonic anhydrase polypeptide as defined herein, and recovering the recombinant carbonic anhydrase polypeptide.

In some aspects, the recombinant carbonic anhydrase polypeptides described herein are for use in an industrial process for capturing CO₂ from a CO₂-containing effluent or gas.

In some aspects, described herein is a process for absorbing CO₂ from a CO₂-containing effluent or gas, the method comprising: contacting the CO₂-containing effluent or gas with an aqueous absorption solution to dissolve the CO₂ into the aqueous absorption solution; and providing the recombinant carbonic anhydrase polypeptide as described herein to catalyze the hydration reaction of the dissolved CO₂ into bicarbonate and hydrogen ions or the reverse reaction.

In some embodiments, the process comprises exposing the recombinant carbonic anhydrase polypeptide variants as described herein to process conditions (e.g., aqueous absorption solution, temperature, and/or pH conditions) that leverage their improved solubility and/or thermostability, resulting in a decrease in the rate or amount of recombinant carbonic anhydrase polypeptide consumption/depletion, as compared to a corresponding process performed with the carbonic anhydrase polypeptide of SEQ ID NO: 1 or 2, or other control carbonic anhydrase polypeptide. Since replenishing the recombinant carbonic anhydrase polypeptide is an operating expense of a CO₂ capture process, decreasing the rate or amount that the enzyme is consumed/depleted by the process would significantly reduce operating costs.

In some embodiments, the decrease in the rate or amount of recombinant carbonic anhydrase polypeptide consumption may result from a decrease in effective concentration of the recombinant carbonic anhydrase polypeptide required to achieve a target level of CO₂ capture, as compared to a corresponding process performed with the carbonic anhydrase polypeptide of SEQ ID NO: it or 2, or other control recombinant carbonic anhydrase polypeptide. More particularly, Example 4 describes that improving the solubility of recombinant carbonic anhydrase as described herein was found to lead to a decrease in the effective concentration of the enzyme required to achieve a given CO₂ capture efficiency, as compared to a control or comparable recombinant carbonic anhydrase having the same or similar thermostability albeit with lower solubility. Without being bound by theory, it is proposed that gains in solubility may reduce the formation of insoluble and/or soluble enzyme aggregates, which are attenuated or inactive in terms of carbonic anhydrase activity. Regardless, the benefit of introducing variants that improve solubility may reduce the amount of enzyme required over time to maintain a given CO₂ capture efficiency. Furthermore, the ability to employ a lower concentration of the recombinant carbonic anhydrase in a CO₂ capture process without sacrificing CO₂ capture performance or efficiency is desirable to potentially reduce operating costs.

In some embodiments, the decrease in the rate or amount of recombinant carbonic anhydrase polypeptide consumption may result from a decrease in the rate or amount of active recombinant carbonic anhydrase polypeptide (i.e., recombinant carbonic anhydrase polypeptides having carbonic anhydrase activity) that is lost or depleted due to aggregation and/or thermal instability, as compared to a corresponding process performed with the carbonic anhydrase polypeptide of SEQ ID NO: 1 or 2, or other control carbonic anhydrase polypeptide. It is shown herein that some thermostable recombinant carbonic anhydrase polypeptides may be prone to aggregation and/or precipitation, particularly at higher temperatures in alkaline carbonate solution (e.g., at 80° C. or higher in 1.45 M K₂CO₃ alpha 0.7). The engineered recombinant carbonic anhydrase polypeptides described herein having improved solubility profiles and/or lower isoelectric points may have greater resistance to precipitation and/or aggregation under conditions regularly encountered in CO₂ capture processes, and thus may reduce operating costs related to enzyme replenishment and/or extra interventions associated with same.

In some embodiments, the recombinant carbonic anhydrase polypeptides described herein are used in combination with an absorption solution comprising at least one absorption compound that aids in the absorption of CO₂. In some embodiments, the absorption solutions described herein may comprise at least one absorption compound such as: (a) a primary amine, a secondary amine, a tertiary amine, a primary alkanolamine, a secondary alkanolamine, a tertiary alkanolamine, a primary amino acid, a secondary amino acid, a tertiary amino acid, dialkylether of polyalkylene glycols, dialkylether or dimethylether of polyethylene glycol, amino acid or a derivative thereof, monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DTPA), methylmonoethanolamine (MMEA), tertiarybutylaminoethoxy ethanol (TBEE), N-2-hydroxyethyl-piperzine 2-amino-2-hydroxymethyl-1,3-propanediol (AHPD), hindered diamine (HDA), bi-(tertiarybutylaminoethoxy)-ethane (BTEE), ethoxyethoethanol-tertiarybutylamine (EEETB), bis-(tertiarybutylaminoethyl)ether, 1,2-bis-(tertiarybutylaminoethoxy)ethane and/or bis-(2-isopropylaminopropyl)ether, or a combination thereof; (b) a primary amine, a secondary amine, a tertiary amine, a primary alkanolamine, a secondary alkanolamine, a tertiary alkanolamine, a primary amino acid, a secondary amino acid, a tertiary amino acid; or a combination thereof; (c) dialkylether of polyalkylene glycols, dialkylether or dimethylether of polyethylene glycol, amino acid or derivative thereof, or a combination thereof; (d) piperazine or derivative thereof, preferably substituted by at least one of alkanol group; (e) monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether; (f) an amino acid or derivative thereof, which is preferably a glycine, proline, arginine, histidine, lysine, aspartic acid, glutamic acid, methionine, serine, threonine, glutamine, cysteine, asparagine, valine, leucine, isoleucine, alanine, tyrosine, tryptophan, phenylalanine, taurine, N-cyclohexyl 1,3-propanediamine, N-secondary butyl glycine, N-methyl N-secondary butyl glycine, diethylglycine, dimethylglycine, sarcosine, methyl taurine, methyl-α-aminopropionicacid, N-(β-ethoxy)taurine, N-(β-aminoethyl)taurine, N-methyl alanine, 6-aminohexanoic acid, potassium or sodium salt of the amino acid, or any combination thereof; (g) a carbonate compound; (h) sodium carbonate, potassium carbonate, or MDEA; (i) sodium carbonate; or (j) potassium carbonate.

In some embodiments, the concentration of the absorption compound in the solution may be between about 0.1 and 10 M, depending on various factors. When the absorption compound is amine-based, the concentration of the amine-based solution may be between about 0.1 and 8 M, and when the absorption compound is amino acid-based, the concentration of the amino acid-based solution may be between about 0.1 and 6 M. When the absorption compound is carbonate based, the pH of the absorption solution may be between about 8 and 12, depending for example on the absorption compound and on the CO₂ loading of the solution.

In some embodiments, the absorption solutions described herein may comprise an absorption compound which is a carbonate compound at concentration from about 0.1 to 3 M, 0.5 to 2.5 M, 0.5 to 2 M, 1 to 2 M, or 1.25 to 1.75 M. In some embodiments, the carbonate compound may be sodium carbonate or potassium carbonate.

In some embodiments, CO₂, capture processes described herein may comprise exposing the recombinant carbonic anhydrase polypeptides described herein to a temperature of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C., and/or to a pH of 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11 at some point during said process (e.g., as part of temperature and/or pH fluctuations within a recurring process thermocycle). In some embodiments, CO₂ capture processes described herein may comprise exposing the recombinant carbonic anhydrase polypeptides described herein to a pH from 8 to 11, 8.5 to 11, 9 to 10.5, or 9 to 10 at some point during said process (e.g., as part of temperature and/or pH fluctuations within a recurring process thermal cycle). Such elevated temperatures and pH, particularly in the context of carbonate solutions, may leverage or exploit the improved solubility and/or thermostability profiles of the recombinant carbonic anhydrase polypeptides described herein to improve CO₂ capture efficiency and/or reduce operating costs.

In some embodiments, the CO₂-containing effluent or gas may comprise between about 0.04 vol % and 80 vol %, 3 vol % and 50 vol %, 5 vol % and 40 vol %, 5 vol % and 35 vol %, or 5 vol % and 30 vol % of CO₂. In some embodiments, the CO₂-containing effluent or gas may comprise N₂, O₂, noble gases, VOCs, H₂O, CO, SOx, NOx compounds, NH₃, mercaptans, H₂S, H₂, heavy metals, dusts, ashes, or any combination thereof. In some embodiments, the CO₂-containing effluent or gas may be derived from natural gas combustion, coal combustion, biogas combustion, biogas upgrading, or natural gas sweetening.

In some embodiments, CO₂ capture processes described herein may employ the carbonic anhydrase polypeptides described herein in the absorption solution at a concentration of about 0.01 to is 50 g/L, 0.05 to 10 g/L, or 0.1 to 4 g/L. In some embodiments, CO₂ capture processes described herein may employ the carbonic anhydrase polypeptides described herein in the absorption solution at a concentration of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/L.

In some embodiments, carbonic anhydrase polypeptides described herein may be prepared in stock or feed solutions (e.g., for use in CO₂ capture processes) comprising a recombinant carbonic anhydrase polypeptide as described herein at a concentration of at least 5, 6, 7, 8, 9, 10, 11, or 12 g/L. In some embodiments, the stock or feed solutions lose less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5% (w/v) of its starting concentration following incubation for 24 hours at 70, 71, 72, 73, 74, 75, 76, 77, 78, 79, 80, 81, 82, 83, 84, 85, 86, 87, 88, 89, or 90° C., for example due to reduced aggregation/precipitation of the enzyme, as compared to a control carbonic anhydrase polypeptide. Such aggregation/precipitation can be determined, for example, as described in Example 1 and Table 1.

In some embodiments, CO₂ capture processes described herein may comprise one or more additional features (e.g., relating to overall CO₂ capture system, absorption unit, desorption unit, separation unit, measurement device, and/or process parameters/conditions) as described in WO/2016/029316 and/or WO/2017/035667.

In various embodiments, the recombinant carbonic anhydrase polypeptides described herein may be employed in CO₂ capture processes as enzymes that are free or dissolved in a solvent, immobilized or entrapped or otherwise attached to particles that are in the absorption solution or to packing material or other structures that are fixed within a reaction chamber. In the case where the recombinant carbonic anhydrase polypeptides described herein may be immobilized with respect to a support material, this may be accomplished by an immobilization technique selected from adsorption, covalent bonding, entrapment, copolymerization, cross-linking, and encapsulation, or combination thereof.

In one scenario, the recombinant carbonic anhydrase polypeptides described herein may be immobilized on a support that is in the form of particles, beads or packing. Such supports may be solid or porous with or without coatings) on their surface. The recombinant carbonic anhydrase polypeptides described herein may be covalently attached to the support and/or the coating of the support, or entrapped inside the support or the coating. In some embodiments, the coating may be a porous material that entraps the recombinant carbonic anhydrase polypeptides described herein within pores and/or immobilizes the enzymes by covalent bonding to the surfaces of the support. In some embodiments, the support material may include nylon, cellulose, silica, silica gel, chitosan, polyacrylamide, polyurethane, alginate, polystyrene, polymethylmetacrylate, magnetic material, sepharose, titanium dioxide, zirconium dioxide and/or alumina, respective derivatives thereof, and/or other materials. In some embodiments, the support material may have a density between about 0.6 g/ml and about 5 g/ml such as a density above 1 g/ml, a density above 2 g/mL, a density above 3 g/mL or a density of about 4 g/mL.

In some scenarios, the recombinant carbonic anhydrase polypeptides described herein may be provided as cross-linked enzyme aggregates (CLEAs) and/or as cross-linked enzyme crystals (CLECs). In the case of using enzymatic particles, including CLEAs or CLECs, the particles may be sized to have a diameter at or below about 17 μm, optionally about 10 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, about 0.2 μm, about 0.1 μm, about 0.05 μm, or about 0.025 μm. The particles may also have a distribution of different sizes.

The scope of the claims should not be limited by the aspects, scenarios, implementations, examples or embodiments set forth in the examples and the description, but should be given the broadest interpretation consistent with the description as a whole.

All patents, published patent applications, and references that are mentioned herein are hereby incorporated by reference. In the case of inconsistencies, the present disclosure will prevail.

### EXAMPLES

Materials and methods are as described in WO/2017/035667 unless otherwise specified.

### Example 1

### Carbonic Anhydrase from Thermovibrio ammonificans Exhibits Marked Decrease in Solubility in an Alkaline Carbonate Buffer at Elevated Temperatures

The solubility wild-type TACA was evaluated by measuring the residual concentration of stock solutions of 6, 9, or 12 g/L of wtTACA (SEQ ID NO: 1) in a 1.45 M K₂CO₃ alpha 0.7 solution, after a 24-hour incubation at 80° C., wherein the alpha is the molar ratio of carbon over potassium. Protein concentration measurements (g/L) were performed using the Bradford method. Samples were centrifuged prior measurement to remove insoluble matter. Interestingly, the solubility of wtTACA was found to drop to only 1.0 g/L after a 24-hour incubation at 80° C. (see Table 1). Without being bound by theory, the dramatic drop in solubility at 80° C. may have been caused by increased exposure of hydrophobic amino acid residues of the wtTACA caused by the higher temperature, resulting in protein aggregation.

In theory, a protein has its lowest solubility at its isoelectric point (pI), which is the pH at which a protein has a net charge of zero. Using the Compute pI/Mw online tool at the ExPASy Bioinformatics Resource Portal (https://web.expasy.org), the theoretical pI of wtTACA is 8.81 (see Table 1), which may not be ideal in terms of solubility for the alkaline conditions employed in CO₂ capture processes. Random mutagenesis and rational design approaches combined with empirical testing were thus employed to engineer and express TACA variants retaining carbonic anhydrase activity yet having progressively lower isoelectric points. The solubilities at 80° C. of several TACA variants having progressively lower isoelectric points are shown in Table 1.

TABLE 1
Solubility of wtTACA and variants after 24 hours at 80° C. in 1.45M K₂CO₃ alpha 0.7

| | | Solubility in 1.45M | | |
|---|---|---|---|---|
| | | K₂CO₃ alpha 0.7 | | |
| | | Conc. of | Conc. | |
| | | starting | after 24 h | |
| | Mutations relative to wtTACA | solution | at 80° C. | |
| | (SEQ ID NO: 1) | (g/L) | (g/L) | pI |
| wtTACA | None | 6.0 | 1.0 | 8.8 |
| SEQ ID NO: 1 | | 9.0 | 1.0 | |
| | | 12.0 | 1.0 | |
| SEQ ID NO: 2 | R156E | 5.0 | 2.0 | 8.3 |
| | | 10.0 | 2.2 | |
| SEQ ID NO: 3 | K27R, N38D, K88R, K116R, N119D, K128R, | n/d | n/d | 7.2 |
| | R156E, E160D, D168E, E192D, E1919D, K203R, | | | |
| | K206R, V216T, L219I | | | |
| SEQ ID NO: 4 | Y77F, V79E, K88E, Y10517, K116E, K128E, | 6 | 4.6 | 6.1 |
| | E137D, E145D, R156E, D168E, Y170F, E195D, | 9 | 7.3 | |
| | E199D, V216T, L219I, K226R | | | |
| SEQ ID NO: 5 | 27K/R; 38N/D; 77Y/F; 79V/E; 88K/R/E; 105Y/F; | n/d | n/d | 6.1-8.3 |
| | 116K/R/E; 119N/D; 128K/R/E; 137E/D, 145E/D; | | | |
| | 156R/E; 160E/D, 168D/E; 170Y/F; 192E/D, | | | |
| | 195E/D; 199E/D; 203K/R; 206K/R; 216V/T; 219L/I; | | | |
| | 226K/R | | | |
| SEQ ID NO: 11 | SEQ ID NO: 4 + S39I, K223I, | 6 | 4.9 | 5.9 |
| | | 9 | 7.4 | |
| | | 12 | 10.7 | |
| SEQ ID NO: 10 | SEQ ID NO: 11 + G130A, T154P, D195E | 6 | 5.0 | 5.9 |
| | | 9 | 9.3 | |
| | | 12 | 12.2 | |

n/d: not determined

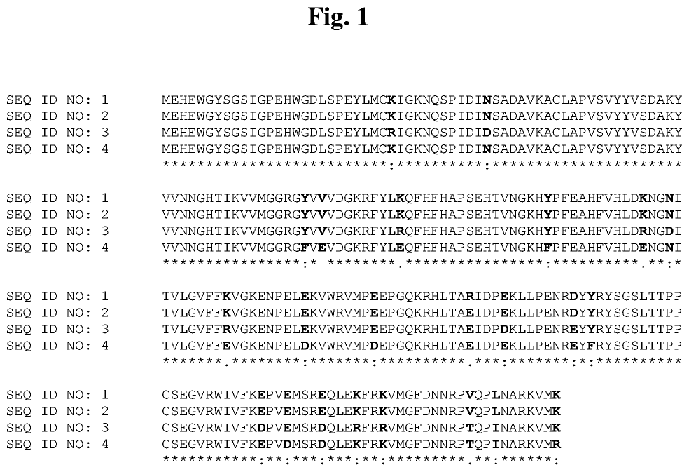

With the goal of finding novel mutations that increase thermostability without negatively impacting solubility, three TACA variants (each having a different pI but all exhibiting carbonic anhydrase activity) were selected and employed as templates for random mutagenesis, as described in Examples 2 and 3. The three templates used for the random mutagenesis were SEQ ID NOs: 2, 3 and 4—see Table 1. SEQ ID NO: 5 is an amalgam of the three templates, and FIG. 1 shows a multiple sequence alignment, of SEQ NOs: 1-4.

### Example 2

### Mutagenesis Screening Based on SEQ ID NO: 2

Large-scale random mutagenesis targeting residues predicted to be solvent-exposed, according to the atomistic protein structure PDB ID NO 4C3T, was performed starting from the TACA variant of SEQ ID NO: 2 having a theoretical isoelectric point of 8.1 Mutated enzymes were expressed in E. coli, purified and characterized as described in WO/2017/035667 for carbonic anhydrase activity and thermostability. Statistics for this round of mutagenesis are shown in Table 2.

TABLE 2

| | |
|---|---|
| Statistics relative to mutagenesis screening in Example | 2 |
| Positions at. the enzyme surface targeted for mutagenesis: | 114 |
| Total number of TACA variants tested: | 8450 |
| Number of TACA variants sequenced: | 371 |
| Number of unique TACA variants identified: | 224 |
| Characterized TACA variants: | 153 |
| Positions with positive variants (improved thermostability | 35 |
| and/or solubility over wtTACA): | |

Solubility of the TACA variants was evaluated by measuring the residual concentration of 5 g/L enzyme in a 1.45 M K₂CO₃ alpha 0.7 solution after a 24-hour incubation at room temperature (RT) or at 70° C., wherein the alpha is the molar ratio of carbon over potassium. Results are shown in Table 3.

Furthermore, to simplify comparison of different TACH variants in terms of their solubility, a single “Solubility score” was assigned to each variant, which takes into account the solubility of that variant at all temperatures tested (RT and 70° C.) in comparison to that of its parent template used as the starting point for mutagenesis of that variant. More particularly, a Solubility Score of 1.0 was assigned when the variant exhibited solubility values comparable to that of its parent template enzyme. A Solubility Score below 1.0 was assigned when the variant exhibited a less favorable solubility profile as compared to its parent template enzyme, while a Solubility Score over 1.0 was assigned when the variant exhibited a more favorable solubility profile as compared to its parent template enzyme. The maximum Solubility Score was set at 1.5, wherein variants assigned to this maximum score exhibited no precipitation/aggregation during solubility testing.

Stability assays were performed by measuring the residual activity for each variant after 3 days exposure in 1.45 M K₂CO₃ alpha 0.7 at 85° C. and then comparing to that of its corresponding parent template enzyme. Results are shown in Table 3, wherein the column labeled “Stability Score” indicates the ratio of those residual activities over that of the corresponding parent template enzyme (SEQ ID NO: 2).

Because both solubility and thermostability are important factors contributing to the effective activity of a particular variant for efficient CO₂ capture over multiple thermal cycles, an Overall Score was assigned for each variant, which was obtained by multiplying both solubility and stability scores. This Overall Score enabled a more meaningful ranking of the different variants for CO₂ capture operations, as compared to ranking individual variants in terms of solubility or stability alone. For example, a variant associated with increased stability but that causes the enzyme to precipitate at higher temperatures may be less attractive than a variant that increases solubility but does not significantly affect stability. In Table 3, the “E156R” mutant having an Overall Score of 0.8 refers to wtTACA, since the amino acid substitution merely reverts the template enzyme back to SEQ ID NO: 1.

TABLE 3
Results of mutagenesis using SEQ ID NO: 2 as starting template

| | Solubility | | | | | |
|---|---|---|---|---|---|---|
| Mutation: | Solubility | Solubility | Solubility | | | |
| SEQ ID | 24 h RT | 24 h 70° C. | Score | Stability | Overall | |
| NO: 2 + | [E] g/L | [E] g/L | (0-1.5) | Score | Score | Comments |
| H3E | 3.9 | 4.0 | 1.0 | 2.4 | 2.4 | |
| G6P | n/d | n/d | n/d | n/d | n/d | Low productivity |
| G6R | 5.0 | 4.2 | 1.5 | n/d | n/d | |
| G9A | 4.2 | 3.1 | 1.5 | 1.0 | 1.5 | |
| G9H | n/d | n/d | n/d | n/d | n/d | Low productivity |
| G9N | 4.6 | 4.7 | 1.5 | n/d | n/d | |
| S10V | 1.1 | 3.4 | 0.5 | 0.9 | 0.5 | |
| S10W | 1.0 | 1.5 | 0.0 | 1.0 | 0.0 | |
| I11L | 2.4 | 3.9 | 1.0 | 1.2 | 1.2 | |
| I11P | 5.7 | 8.3 | 1.5 | 1.5 | 2.3 | |
| I11V | 3.2 | 5.0 | 1.0 | 0.7 | 0.7 | |
| I11Y | 5.9 | 5.0 | 1.5 | 1.0 | 1.5 | |
| G12D | 4.5 | 3.7 | 1.5 | 1.1 | 1.7 | |
| G12R | 3.8 | 4.9 | 1.0 | 1.4 | 1.4 | |
| H15L | 3.1 | 3.9 | 1.0 | 1.4 | 1.4 | |
| G17Y | 4.6 | 3.9 | 1.5 | 0.5 | 0.8 | |
| D18I | 3.6 | 1.8 | 0.3 | 1.8 | 0.5 | |
| D18L | 3.9 | 2.1 | 0.5 | 1.6 | 0.8 | |
| D18R | 3.1 | 3.6 | 1.0 | 1.2 | 1.2 | |
| D18S | 4.7 | 3.5 | 1.5 | 1.3 | 2.0 | |
| S20F | 4.4 | 2.8 | 0.8 | n/d | n/d | |
| S20I | 4.6 | 2.8 | 0.8 | n/d | n/d | |
| S20K | 4.2 | 5.2 | 1.5 | n/d | n/d | |
| S20L | 4.6 | 3.1 | 1.5 | n/d | n/d | |
| S20T | 3.2 | 4.5 | 1.0 | n/d | n/d | |
| S20V | 3.2 | 2.7 | 0.5 | n/d | n/d | |
| L24I | 5.8 | 4.4 | 1.5 | 1.7 | 2.6 | |
| L24M | 7.2 | 4.2 | 1.5 | 1.0 | 1.5 | |
| L24W | 3.9 | 4.4 | 1.0 | 0.7 | 0.7 | |
| L24F | 2.6 | 4.2 | 1.0 | 0.9 | 0.9 | |
| L24V | 4.2 | 4.4 | 1.5 | 1.1 | 1.7 | |
| M25F | 3.6 | 3.9 | 1.0 | 1.3 | 1.3 | |
| K27F | 1.7 | 4.5 | 0.5 | 0.6 | 0.3 | |
| K27Q | 3.0 | 5.0 | 1.0 | 0.3 | 0.3 | |
| K27V | 3.0 | 4.9 | 1.0 | 0.7 | 0.7 | |
| K27R | 2.0 | 4.5 | 0.5 | n/d | n/d | |
| D36L | 2.3 | 0.0 | 0.0 | n/d | n/d | |
| N38A | 2.6 | 3.6 | 1.0 | 0.7 | 0.7 | |
| N38D | 2.2 | 3.6 | 1.0 | 1.3 | 1.3 | |
| N38R | 2.3 | 4.3 | 1.0 | 1.3 | 1.3 | |
| N38T | 2.1 | 3.3 | 1.0 | 1.9 | 1.9 | |
| N38V | 2.4 | 4.4 | 1.0 | 1.0 | 1.0 | |
| N38W | n/d | n/d | 0.0 | n/d | 0.0 | Not soluble in |
| | | | | | | 1.45M K₂CO₃ alpha |
| | | | | | | 0.7 |
| S39H | 4.4 | 3.5 | 1.5 | 1.5 | 2.3 | |
| S39I | 4.5 | 4.5 | 1.5 | 3.5 | 5.3 | |
| S39L | 4.4 | 4.7 | 1.5 | 3.1 | 4.7 | |
| S39R | 4.7 | 3.4 | 1.5 | 1.3 | 2.0 | |
| S39W | 4.5 | 3.6 | 1.5 | 0.9 | 1.4 | |
| K44L | 1.4 | 4.6 | 0.5 | 1.0 | 0.5 | |
| K44R | 4.8 | 2.1 | 0.8 | n/d | n/d | |
| K44W | 4.0 | 2.6 | 0.5 | 0.2 | 0.1 | |
| A48L | 3.3 | 2.7 | 0.5 | 1.3 | 0.7 | |
| A48Q | 3.5 | 4.5 | 1.0 | 1.5 | 1.5 | |
| A48R | 3.3 | 3.5 | 1.0 | 0.6 | 0.6 | |
| A48T | 2.9 | 4.7 | 1.0 | 1.1 | 1.1 | |
| S51A | n/d | n/d | n/d | n/d | n/d | Low productivity |
| S51D | 2.8 | 4.6 | 1.0 | 1.5 | 1.5 | |
| S51E | 3.4 | 4.0 | 1.0 | 1.4 | 1.4 | |
| S51F | 4.2 | 4.7 | 1.0 | 0.9 | 1.4 | |
| S51M | 3.2 | 4.4 | to | 1.1 | 1.1 | |
| S51P | 3.2 | 3.8 | 1.0 | 1.5 | 1.5 | |
| S51R | 3.0 | 3.7 | 1.0 | 0.7 | 0.7 | |
| S51T | 3.4 | 3.7 | 1.0 | 0.8 | 0.8 | |
| Y53T | 3.2 | 3.8 | 1.0 | 0.6 | 0.6 | |
| V55E | 3.6 | 4.7 | 1.0 | n/d | n/d | |
| S56P | 3.2 | 1.3 | 0.3 | 1.1 | 0.3 | |
| Y60F | 4.0 | 5.2 | 1.0 | 0.7 | 0.7 | |
| Y60H | 3.2 | 4.8 | 1.0 | 0.5 | 0.5 | |
| N64T | 4.1 | 2.2 | 0.8 | 1.1 | 0.8 | |
| N64V | 1.5 | 4.3 | 0.5 | 0.9 | 0.5 | |
| G65K | 2.3 | 4.9 | 1.0 | 0.3 | 0.3 | |
| K69V | 4.2 | 2.3 | 0.8 | 0.0 | 0.0 | |
| G73E | 3.1 | 4.4 | 1.0 | 1.3 | 1.3 | |
| G73L | 4.2 | 3.7 | 1.5 | 0.8 | 1.2 | |
| G73R | 3.0 | 1.8 | 0.3 | n/d | n/d | |
| V79L | 3.1 | 4.5 | 1.0 | 1.2 | 1.2 | |
| V79R | 3.4 | 4.6 | 1.0 | 1.0 | 1.0 | |
| V79W | 5.0 | 4.9 | 1.5 | 1.1 | 1.7 | |
| K88I | 4.5 | 4.7 | 1.5 | 1.3 | 2.0 | |
| K88L | 4.6 | 4.8 | 1.5 | 1.2 | 1.8 | |
| K88R | 4.1 | 2.9 | 0.8 | 1.0 | 0.8 | |
| K88V | 4.5 | 4.5 | 1.5 | 1.2 | 1.8 | |
| K88T | 4.5 | 4.5 | 1.5 | 1.0 | 1.5 | |
| N101R | 2.8 | 0.9 | 0.0 | n/d | 0.0 | |
| G102A | 4.3 | 2.3 | 0.8 | 0.5 | 0.4 | |
| G102R | 2.1 | 4.0 | 1.0 | 0.6 | 0.6 | |
| N119C | 2.8 | 4.3 | 1.0 | 0.9 | 0.9 | |
| N119M | 2.1 | 3.8 | 1.0 | 1.3 | 1.3 | |
| N119W | 2.6 | 2.6 | 0.5 | 0.9 | 0.5 | |
| K128T | 4.3 | 3.3 | 1.5 | 0.6 | 0.9 | |
| V179I | 1.8 | 4.1 | 0.0 | n/d | 0.0 | Precipitated stock |
| | | | | | | enzyme |
| V129Y | n/d | n/d | 0.0 | n/d | 0.0 | Precipitated stock |
| | | | | | | enzyme |
| K138E | 1.7 | 4.9 | 0.5 | 1.1 | 0.6 | |
| K138H | 1.5 | 4.6 | 0.5 | 1.0 | 0.5 | |
| K138L | 2.1 | 4.3 | 1.0 | 1.1 | 1.1 | |
| K138R | 2.9 | 4.0 | 1.0 | 1.0 | 1.0 | |
| K138V | 1.6 | 4.4 | 0.5 | 0.6 | 0.3 | |
| E146R | 3.7 | 2.3 | 0.5 | 1.0 | 0.5 | |
| G148F | 2.0 | 4.6 | 0.5 | 1.8 | 0.9 | |
| G148V | 4.5 | 4.5 | 1.5 | 0.6 | 0.9 | |
| G148W | 4.0 | 4.5 | 1.0 | 1.9 | 1.9 | |
| Q149I | 4.8 | 3.6 | 1.5 | 0.9 | 1.4 | |
| Q149L | 3.9 | 1.9 | 0.3 | 0.9 | 0.2 | |
| Q149T | 2.9 | 3.5 | 1.0 | 1.0 | 1.0 | |
| T154P | 3.3 | 3.2 | 1.0 | 1.7 | 1.7 | |
| T154D | 3.4 | 3.7 | 1.0 | 1.4 | 1.4 | |
| T154V | 0.6 | 2.5 | 0.0 | 1.4 | 0.0 | |
| E156R | 4.0 | 4.2 | 1.0 | 0.8 | 0.8 | wtTACA |
| | | | | | | SEQ ID NO: 1 |
| R156V | 2.8 | 4.3 | 1.0 | 1.1 | 1.1 | |
| D158R | 2.6 | 4.7 | 1.0 | 1.0 | 1.0 | |
| D158Y | 1.5 | 4.2 | 0.5 | 1.3 | 0.7 | |
| E160D | 4.0 | 4.7 | 1.0 | 0.5 | 0.5 | |
| E160Q | 2.1 | 4.7 | 1.0 | 1.3 | 1.3 | |
| E165L | 1.5 | 3.0 | 0.3 | 1.0 | 0.3 | |
| E165V | 1.7 | 2.4 | 0.3 | 1.2 | 0.3 | |
| N166E | 4.0 | 3.5 | 1.0 | 1.3 | 1.3 | |
| N166G | 2.9 | 4.5 | 1.0 | 1.0 | 1.0 | |
| N166L | 4.0 | 5.0 | 1.0 | 1.0 | 1.0 | |
| N166V | 4.7 | 4.9 | 1.5 | 1.0 | 1.5 | |
| R167L | 2.1 | 4.5 | 1.0 | 1.8 | 1.8 | |
| D168F | 4.5 | 4.6 | 1.5 | 0.5 | 0.8 | |
| D168R | 4.9 | 4.8 | 1.5 | 1.1 | 1.7 | |
| D168W | 4.8 | 4.9 | 1.5 | 0.8 | 1.2 | |
| S182R | 3.4 | 4.6 | 1.0 | 0.9 | 0.9 | |
| S182T | 3.1 | 4.1 | 1.0 | 0.9 | 0.9 | |
| E199A | 3.1 | 4.1 | 1.0 | 1.6 | 1.6 | |
| E199D | 3.3 | 4.5 | 1.0 | 0.9 | 0.9 | |
| E199K | 4.5 | 4.8 | 1.5 | 0.9 | 1.4 | |
| K203T | 2.7 | 4.3 | 1.0 | 1.0 | 1.0 | |
| K203V | 2.9 | 4.5 | 1.0 | 1.1 | 1.1 | |
| G209S | 0.9 | 1.7 | 0.0 | n/d | 0.0 | |
| F210H | 4.5 | 4.7 | 1.5 | n/d | n/d | |
| F210M | 3.9 | 4.5 | 1.0 | n/d | n/d | |
| D211H | n/d | n/d | 0.0 | n/d | 0.0 | Precipitated stock |
| | | | | | | enzyme |
| D211F | 1.6 | 0.0 | 0.0 | n/d | 0.0 | |
| D211W | 4.2 | 0.0 | 0.0 | n/d | 0.0 | |
| N213M | 2.9 | 5.1 | 1.0 | 0.4 | 0.4 | |
| P215R | 0.7 | 0.0 | 0.0 | n/d | n/d | |
| P218N | 3.6 | 4.3 | 1.0 | 0.5 | 0.5 | |
| N220Y | 3.5 | 4.2 | 1.0 | 0.9 | 0.9 | |
| K223I | 3.9 | 4.6 | 1.0 | 1.7 | 1.7 | |
| K223L | 4.0 | 4.7 | 1.0 | 2.4 | 2.4 | |
| K223V | 3.3 | 4.2 | 1.0 | 1.6 | 1.6 | |
| K226P | 5.0 | 4.7 | 1.5 | 0.0 | 0.0 | |

“Low productivity”: variant enzyme was not expressed in sufficient quantities to enable characterization; “n/d”: not determined.

### Example 3

### Mutagenesis Screening Based on SEQ ID NOs: 3 and 4

The TACA variant of SEQ ID NO: 3 having a theoretical isoelectric point of 7.16 was constructed by introducing the following 15 mutations relative to wtTACA: K₂₇R, N38D, K88R, K116R, N119D, K128R, R156E, E160D, D168E, E192D, E199D, K₂₀₃R, K206R, V216T, and L219I (see FIG. 1 and Table 1). In parallel, the TACA variant of SEQ ID NO: 4 having a theoretical isoelectric point of 6.06 was constructed by introducing the following 16 mutations relative to wtTACA: Y77F, V79E, K88E, Y105F, K116E, K128E, E137D, E145D, R156E, D168E, Y170F, E195D, E199D, V216T, L219I, and K₂₂₆R (see FIG. 1 and Table 1).

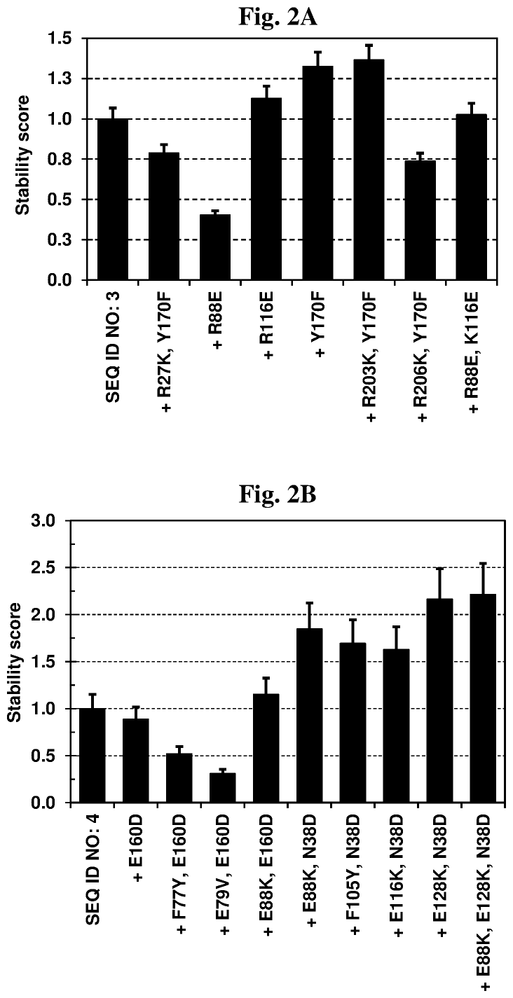

Mutated enzymes were expressed in E. coli, purified and characterized for carbonic anhydrase activity, solubility and thermostability as described above in Examples 1 and 2. Examples of stability testing results for variants generated from SEQ ID NO: 3 and 4 are shown in FIGS. 2A and 2B, respectively.

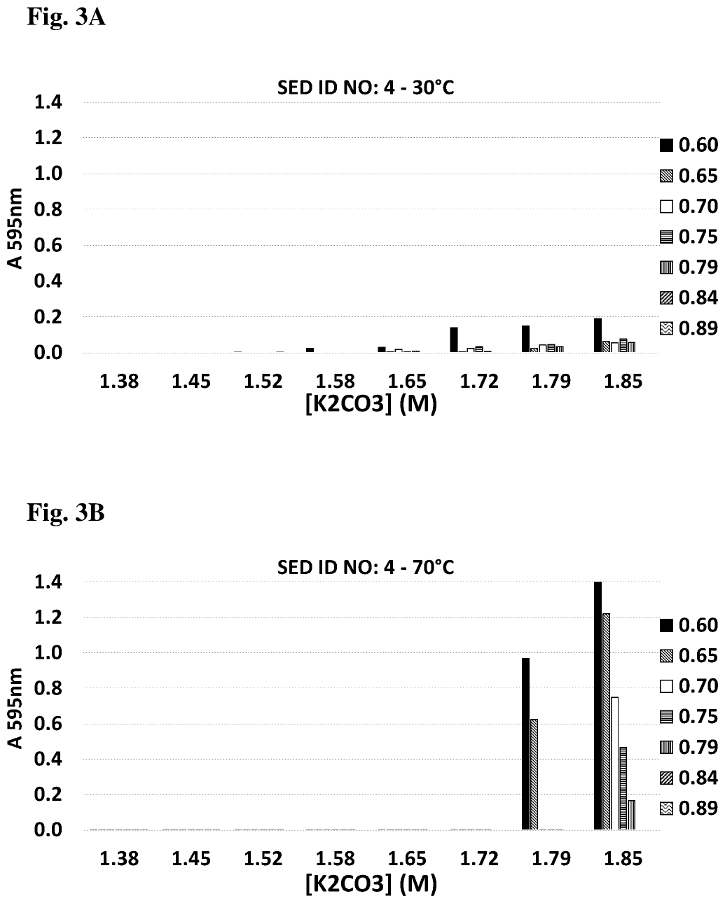

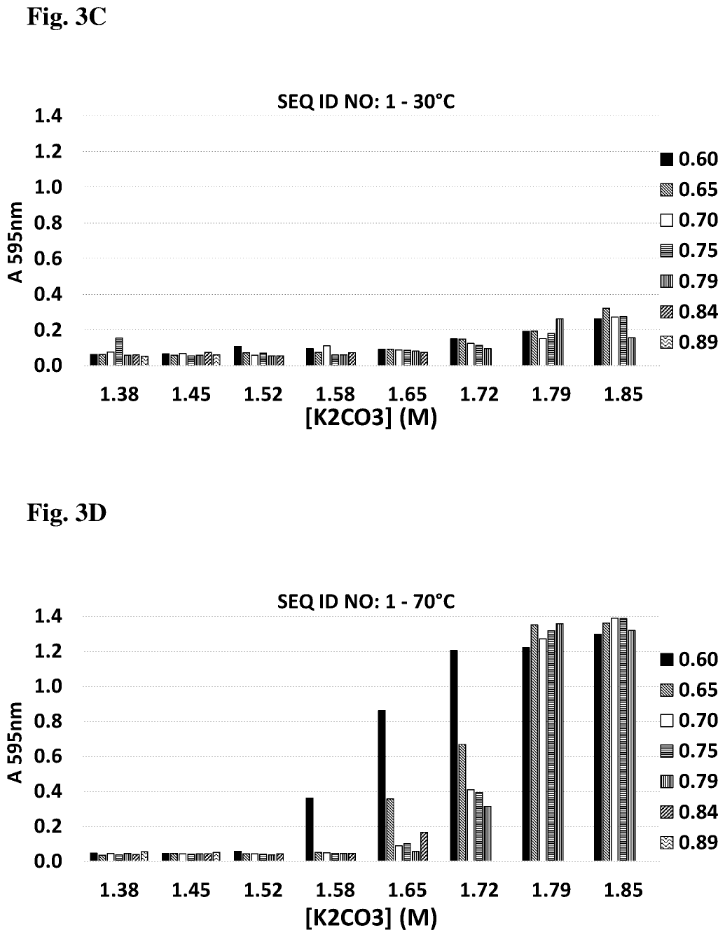

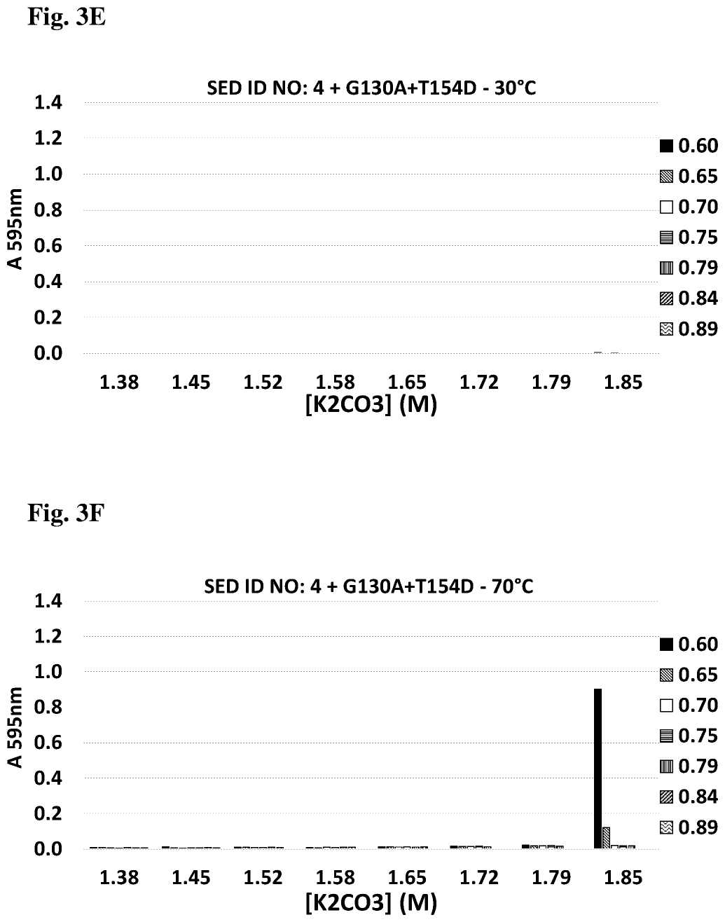

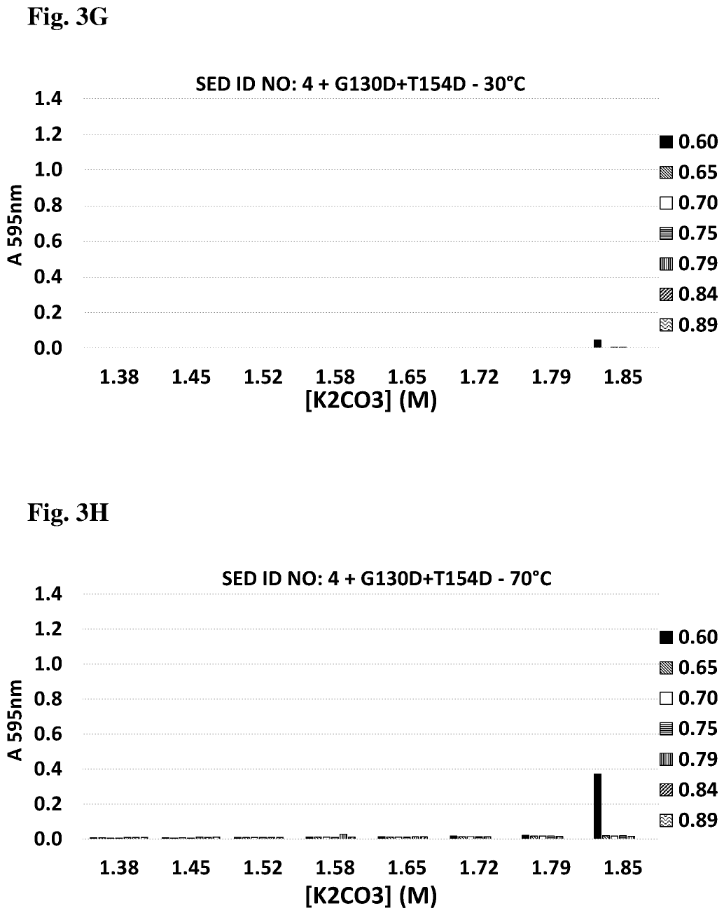

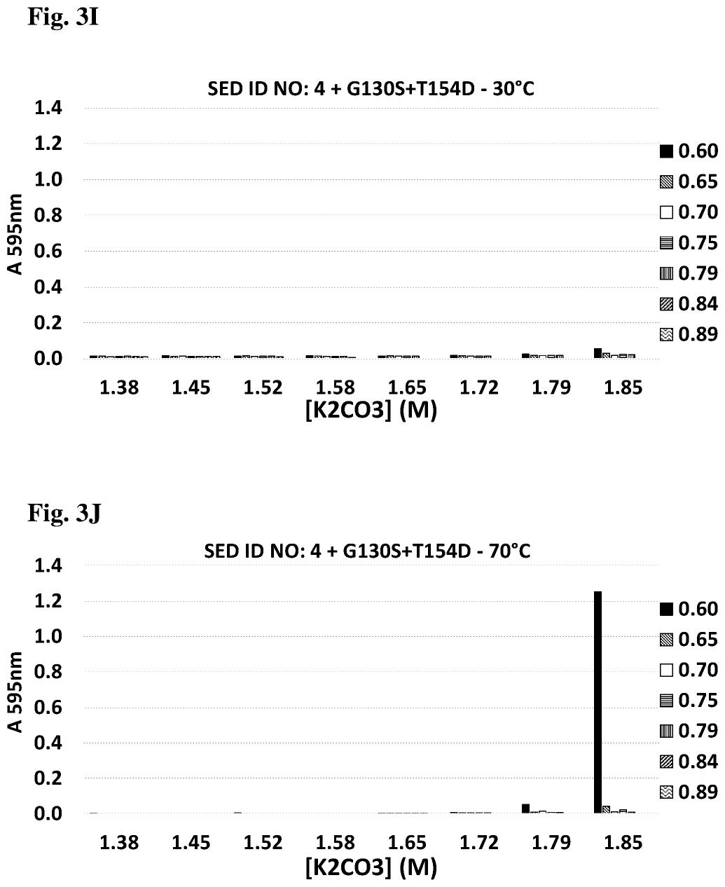

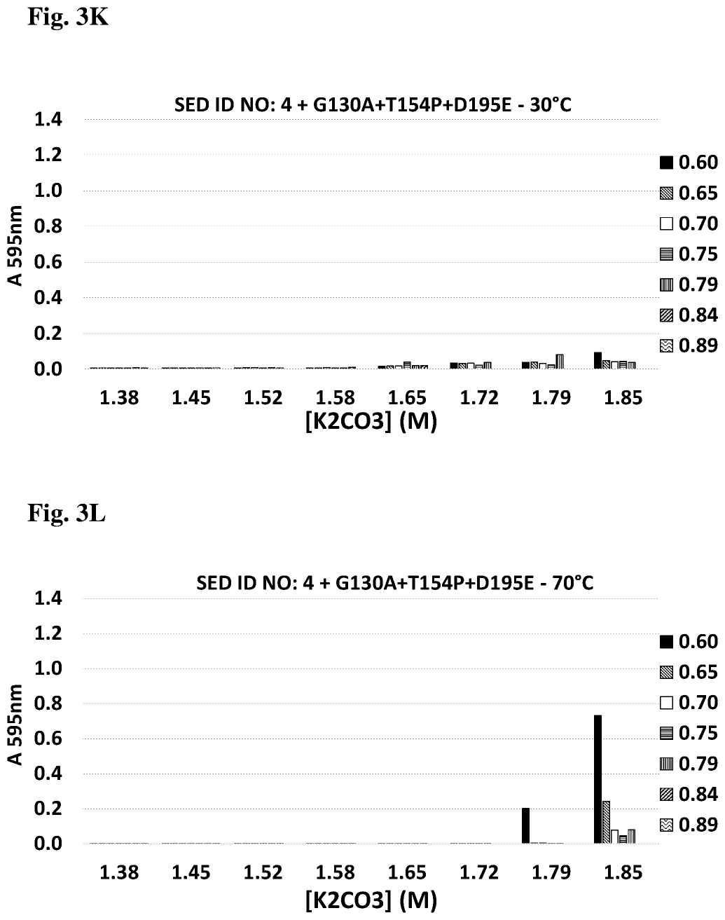

Furthermore, given the relatively higher baseline solubilities of both template enzymes used as starting points in Example 3 over the template used in Example 2, more stringent titration solubility testing was employed to identify amino acid substitutions associated with further increased solubility. The titration solubility testing was performed by measuring the turbidity of multiple solutions containing 2 g/L enzyme. The solutions ranged from 1.38 to 1.85 M K₂CO₃ with alpha varying from 0.60 to 0.89. A low turbidity (near zero) indicates a soluble enzyme, while a high turbidity indicates enzyme aggregation. Examples of titration solubility testing results are shown in FIG. 3.

Results of the characterized TACA variants generated from the templates of SEQ NOs: 3 and 4 are shown in Tables 4 and 5. Of note, the “Solubility Scores” in Tables 4 and 5 differ from those in Example 2 in that the scores were modified to include data from both solubility tests (the ones described in Example 2 and the further titration solubility testing described above). As for Table 3, a Solubility Score of 1.0 was assigned when the variant exhibited solubility values comparable to that of its parent template enzyme. A Solubility Score below 1.0 was assigned when the variant exhibited a less favorable solubility profile as compared to its parent template enzyme, while a Solubility Score greater than 1.0 was assigned when the variant exhibited a more favorable solubility profile as compared to its parent template enzyme. The maximum Solubility Score was set at 1.5, wherein variants assigned this maximum score exhibited no detectable precipitation/aggregation during all solubility testing.

Moreover, characterization of the TACA variants in Tables 4 and 5 enabled the identification of individual amino acid substitutions having a positive effect on enzyme stability and/or solubility. To be considered as having a positive effect (“AA with positive effect on solubility” or “AA with positive effect on stability”), the score of the variant carrying the mutation must be at least 10% higher than one without this mutation. As an example, mutant “SEQ ID NO: 4+N38D+E128K+D137E” has a stability score of 3.26 while the mutant “SEQ ID NO: 4+N38D+E128K+D137E+T154D” has a stability score of 4.89. The difference between those two mutants is T154D for a score increase of 50%. Thus, T154D is a mutation with a positive effect on stability.

Finally, Overall Scores were assigned to each variant by multiplying both solubility and stability scores. Overall Scores above 1.0 indicated a positive effect of the amino acid mutations on enzyme solubility and/or stability.

TABLE 4
Results of mutagenesis using SEQ ID NO: 3 as starting template

| | Solubility | | | | |
|---|---|---|---|---|---|
| Mutation: | Solubility | | Stability | | |
| SEQ ID NO: 3 | Score | AA with positive | Stability | AA with positive | Overall |
| + | (0-1.5) | effect on solubility | Score | effect on stability | Score |
| — | 1.0 | — | 1.00 | — | 1.0 |
| R271K + Y170F | 1.0 | — | 0.79 | — | 0.8 |
| R88E | n/d | — | 0.40 | — | n/d |
| R116E | 1.5 | 116E | 1.13 | 116E | 1.7 |
| R128E | 0 | — | 3.57 | 128E | 0.0 |
| Y170F | 0.375 | — | 1.33 | 170F | 0.5 |
| R203K + Y170F | 1.0 | — | 1.36 | — | 1.4 |
| R206K + Y170F | 1.0 | — | 0.74 | — | 0.7 |
| R88E + K116E | 1.5 | 116E | 1.03 | 116E | 1.5 |
| R88E + R128E | 0 | — | 2.68 | 128E | 0.0 |

n/d: Not determined.

TABLE 5
Results of mutagenesis using SEQ ID NO: 4 as starting template

| | Solubility | | | | |
|---|---|---|---|---|---|
| | Solubility | | Stability | | |
| Mutation: | Score | AA with positive | Stability | AA with positive | Overall |
| SEQ ID NO: 4 + | (0-1.5) | effect on solubility | Score | effect on stability | Score |
| — | 1.0 | — | 1.00 | — | 1.0 |
| H15L + N38D + E128K | 1.0 | — | 2.77 | 15L | 2.8 |
| E22P | 0 | — | 0.54 | — | 0.0 |
| M25F + N38D + E128K | 1.0 | — | 2.69 | 25F | 2.7 |
| N38D | 1.0 | — | 2.29 | 38D | 2.3 |
| N38D + E116K | 0.75 | — | 1.63 | — | 1.2 |
| - | 2.4 | | | | |
| N38D + E128K | 1.0 | — | 2.44 | 38D | 2.4 |
| N38D + E128K + D137E | 1.0 | — | 3.26 | 137E | 3.3 |
| N38D +E128K + | 1.0 | — | 4.89 | 154D | 4.9 |
| D137E + T154D | | | | | |
| N38D + E128K + | 1.0 | — | 4.02 | 154P | 4.0 |
| D137E + T154P | | | | | |
| N38D + E128K + D145E | 1.0 | — | 3.91 | 145E | 3.9 |
| N38D + E128K + D195E | 1.0 | — | 2.48 | — | 2.5 |
| N38D + E128K + D199A | 1.0 | — | 2.43 | — | 2.4 |
| N38D + E128K + E160Q | 1.0 | — | 2.33 | — | 2.3 |
| N38D + E128K + E168D | 1.0 | — | 2.41 | — | 2.4 |
| N38D + E128K + G148W | 0.9 | — | 3.30 | 148W | 3.0 |
| N38D + E128K + I219L | 1.0 | — | 2.58 | — | 2.6 |
| N38D + E128K + R167L | 1.0 | — | 2.69 | 167L | 2.7 |
| N38D + E128K + R226K | 1.0 | — | 2.38 | — | 2.4 |
| N38D + E128K + T216V | 1.0 | — | 2.13 | — | 2.1 |
| N38D + N38D + E88K | 1.0 | — | 2.80 | 88I | 2.8 |
| N38D + E88K | 1.0 | — | 1.85 | — | 1.8 |
| N38D + E88K + E128K | 1.0 | — | 2.21 | — | 2.2 |
| N38D + F105Y | 0.75 | — | 1.69 | — | 1.3 |
| N38T + E128K | 1.0 | — | 1.59 | 38T | 1.6 |
| S39I | 1.5 | 39I | 3.12 | 39I | 4.7 |
| S39I + D195E | 1.5 | ** | 3.79 | 195E | 5.7 |
| S39I + E128K + | 1.0 | — | 4.74 | ** | 4.7 |
| T154D + K223I | | | | | |
| (SEQ ID NO: 9) | | | | | |
| S39I + G130A + | 1.5 | ** | 5.88 | ** | 8.8 |
| T154D + K223I | | | | | |
| (SEQ ID NO: 9) | | | | | |
| S39I + G130A + | 1.5 | ** | 8.26 | ** | 12.4 |
| T154D + K223L | | | | | |
| (SEQ ID NO: 10) | | | | | |
| S39I + G130A + T154P + | 1.5 | ** | 3.81 | ** | 5.7 |
| D195E + K223I | | | | | |
| (SEQ ID NO: 11) | | | | | |
| S39I + G130A + T154P + | 1.4 | ** | 9.21 | ** | 12.9 |
| D195E + K223L | | | | | |
| (SEQ ID NO: 7) | | | | | |
| S39I + K223I | 1.5 | 39I | 4.17 | 223I | 6.2 |
| S39I + K223L | 1.5 | 39I | 4.83 | 223L | 7.2 |
| (SEQ ID NO: 6 | | | | | |
| S39L | 1.5 | 39L | 3.22 | 39L | 4.8 |
| S39L + K223I | 1.5 | 39L | 3.17 | — | 4.8 |
| S39L + K223L | 1.3 | 39L | 4.16 | 223L | 5.4 |
| F77Y + E160D | 1.0 | — | 0.52 | — | 0.5 |
| E79V + E160D | 1.0 | — | 0.31 | — | 0.3 |
| E88K + E160D | 0.5625 | — | 1.15 | — | 0.6 |
| E128K | 1.0 | — | 1.11 | 128K | 1.1 |
| E128K + D195E | 1.0 | — | 1.82 | 195E | 1.8 |
| G130A | 0.75 | — | 1.91 | 130A | 1.4 |
| G130A + T154D | 1.3 | 154D | 4.11 | 130A, 154D | 5.3 |
| G130A + T154P | n/d | — | 1.40 | — | n/d |
| G130A + T154P + D195E | 1.0 | — | 3.41 | 195E | 3.4 |
| G130D | 1.0 | — | 2.11 | 130D | 2.1 |
| G130D + T154D | 1.4 | 154D | 3.24 | 130D, 154D | 4.5 |
| G130D + T154P | n/d | — | 1.36 | — | n/d |
| G130E | 1.0 | — | 1.62 | 130E | 1.6 |
| G130F | 0 | — | 1.57 | 130F | 0.0 |
| G130H | 0.75 | — | 1.28 | 130H | 1.0 |
| G130I | n/d | — | 0.92 | — | n/d |
| G130K | 1.0 | — | 1.48 | 130K | 1.5 |
| G130L | n/d | — | 0.74 | — | n/d |
| G130M | n/d | — | 0.70 | — | n/d |
| G130N | n/d | — | 0.81 | — | n/d |
| G130P | n/d | — | 0.00 | — | 0 |
| G130Q | 0 | — | 1.69 | 130Q | 0.0 |
| G130R | 0.75 | — | 1.22 | 130R | 0.9 |
| G130S | 0.75 | — | 3.42 | 130S | 2.6 |
| G130S + T154D | 1.3 | 154D | 2.36 | — | 3.1 |
| G130S + 154P | n/d | — | 1.96 | — | n/d |
| G130T | 0.75 | — | 1.25 | 130T | 0.9 |
| G130V | 0 | — | 3.30 | 130V | 0.0 |
| G130W | 0 | — | 3.13 | 130W | 0.0 |
| G130Y | 0 | — | 3.40 | 130Y | 0.0 |
| T154D | 1.0 | — | 2.46 | 154D | 2.5 |
| T154E | n/d | — | 0.96 | — | n/d |
| T154K | n/d | — | 1.44 | 154K | n/d |
| T154P | 1.0 | — | 2.08 | 154P | 2.1 |
| T154S | n/d | — | 0.00 | — | 0 |
| T154V | 0 | — | 2.17 | 154V | 0.0 |
| E160D | 1.0 | — | 0.89 | — | 0.9 |
| D195E + K2231 | 1.3 | ** | 2.94 | 195E | 3.8 |
| K223I | 1.3 | 223I | 2.41 | 223I | 3.1 |
| K223L | 1.0 | — | 3.09 | 223L | 3.1 |

** Not possible to identify the individual amino acid causing the increase in stability or solubility.
n/d: Not determined.

### Example 4

### Combinations of Multiple Beneficial Mutations Result in Variants that Surpass wtTACA in Terms of Stability and Solubility

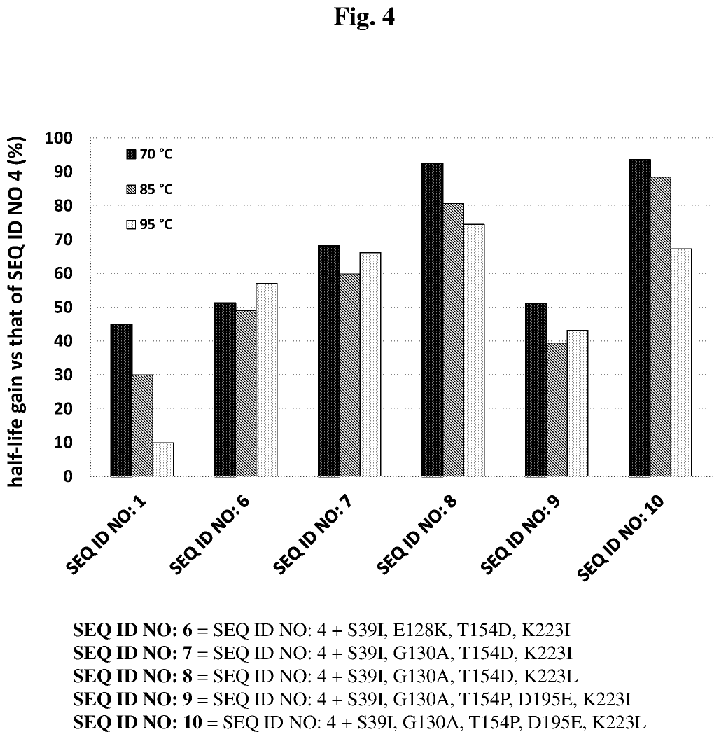

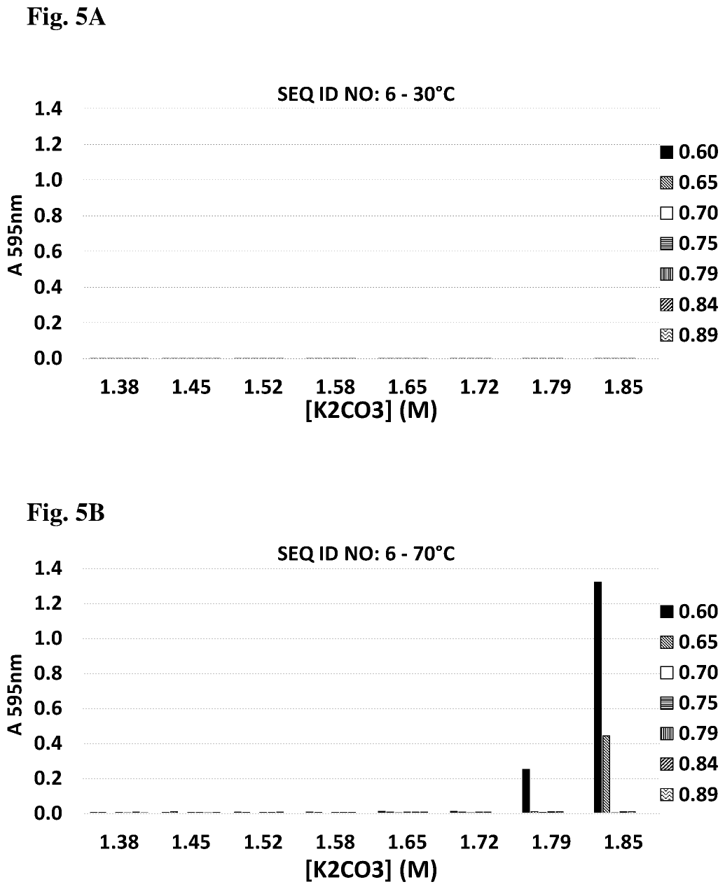

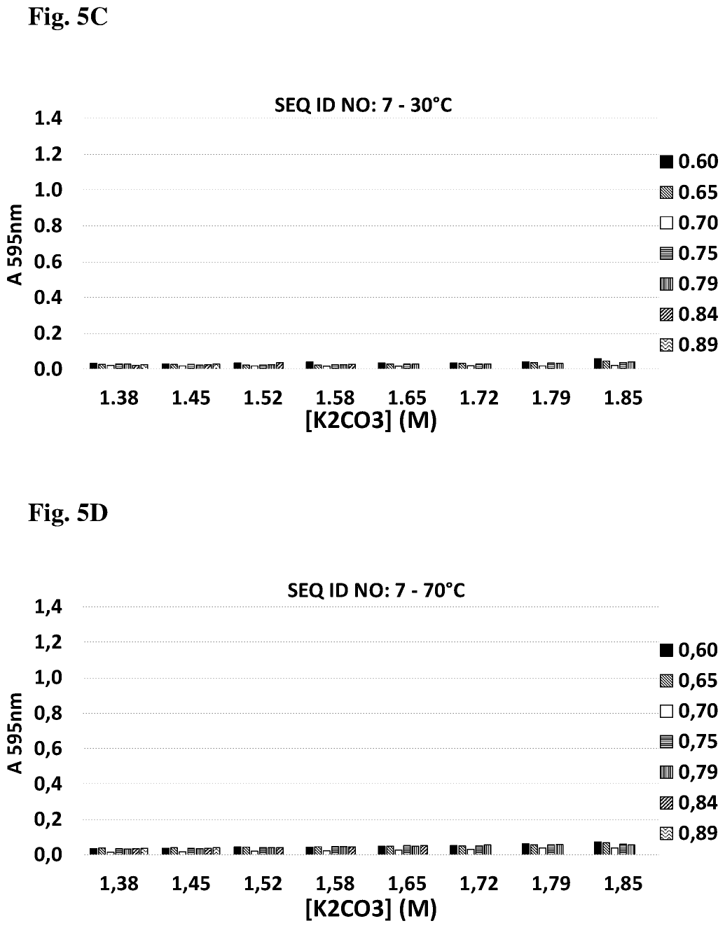

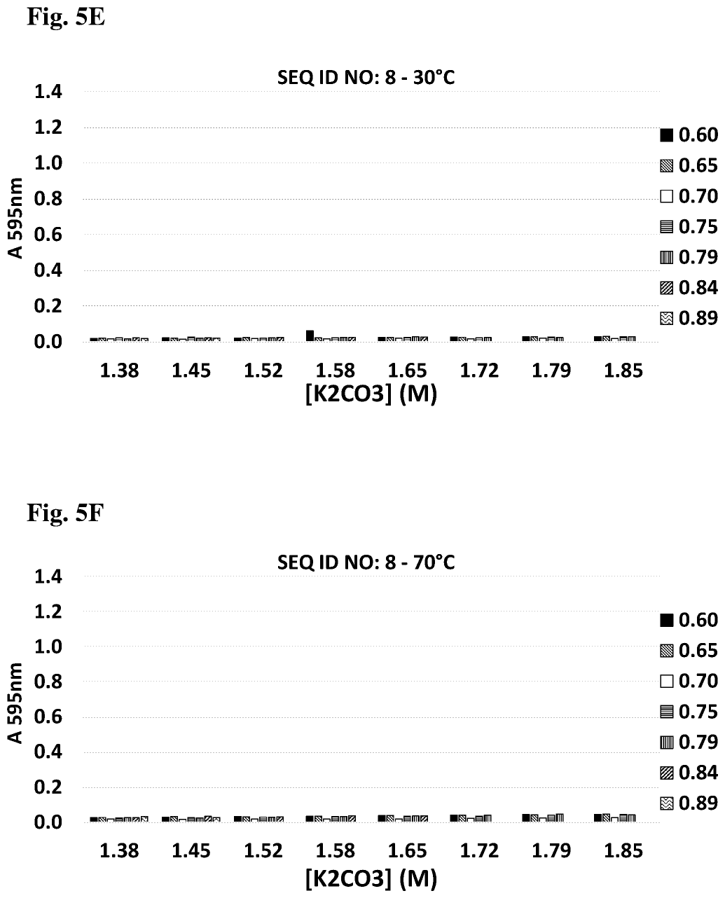

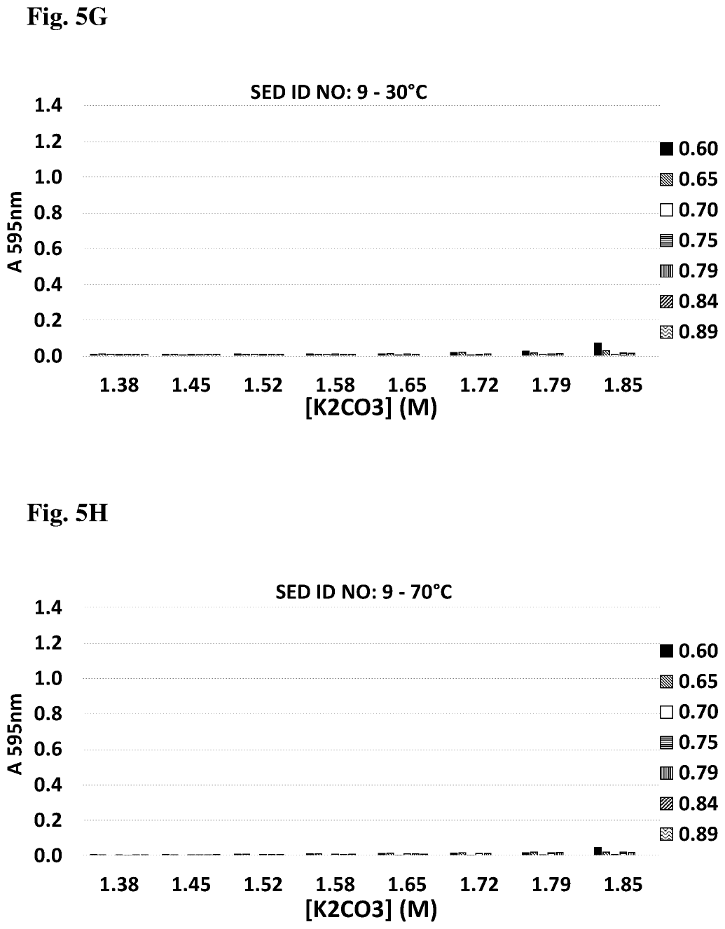

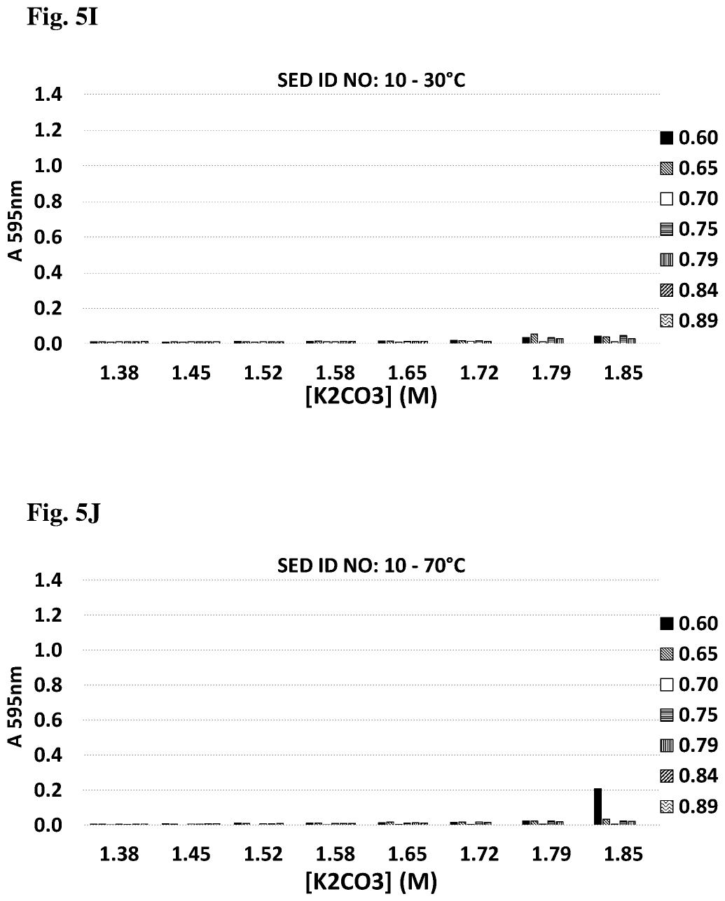

In general, it was found that individual mutations having a beneficial effect in terms of solubility and/or thermostability in one template also had the same beneficial effect when the same mutations were introduced in another template. Furthermore, it was found that combining several mutations having beneficial effects on solubility and/or stability enabled the production of recombinant enzymes having greater thermostability and/or improved solubility than wtTACA in alkaline carbonate CO₂ capture solutions—see FIGS. 4 and 5, and SEQ ID NOs: 6 and 7 in Table 1.

Some beneficial variants were found to improve both thermostability and solubility, while other beneficial variants were found to improve either thermostability or solubility. Variants associated with improved thermostability provide a clear benefit to CO₂ capture processes, for example by reducing the amount of enzyme required over time to maintain a given CO₂ capture efficiency. Interestingly, variants associated with improved solubility provided a similar benefit to CO₂ capture processes. More particularly, improving the solubility of an enzyme may reduce the effective concentration of the enzyme required to achieve a given CO₂ capture efficiency, as compared to an enzyme having the same thermostability albeit with lower solubility. Without being bound by theory, it is proposed that gains in solubility may reduce the formation of soluble enzyme aggregates, which are attenuated or inactive in terms of carbonic anhydrase activity. Regardless, the benefit of introducing variants that improve solubility may reduce the amount of enzyme required over time to maintain a given CO₂ capture efficiency.

What is claimed is:

1. A recombinant carbonic anhydrase polypeptide having carbonic anhydrase activity comprising an amino acid sequence having at least 60% identity with SEQ ID NO: 5 and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223, wherein said recombinant carbonic anhydrase polypeptide has increased solubility and/or increased thermostability as compared to a corresponding carbonic anhydrase polypeptide lacking said one or more amino acid differences.

2. The recombinant carbonic anhydrase polypeptide of claim 1 having an isoelectric point (pI) below that of SEQ ID NO: 2, 3, or 4.

3. The recombinant carbonic anhydrase polypeptide of claim 1 having a pI of below 8.3, 8.2. 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, or 4.5.

4. The recombinant carbonic anhydrase polypeptide of claim 1 having a pI of 4 to 8, 4.5 to 7.5, 5 to 7, 5.5 to 6.5, or 5 to 6.

5. The recombinant carbonic anhydrase polypeptide of claim 1, comprising the residue(s): 3E; 6R; 9A or 9N; 11L, 11P, or 11Y; 15L; 17Y; 18I, 18L, 18R, or 18S; 20K or 20L; 24I, 24M, or 24V; 25F; 27R; 38D, 38R, or 38T; 39H, 39I, 39L, 39R, or 39W; 48L, 48Q, or 48T; 51D, 51E, 51F, 51M, or 51P; 64T; 73E or 73L; 77F; 79E, 79L or 79W; 88E, 88I, 88L, 88R, 88T, or 88V; 105F; 116E or 116R; 119D or 119M; 128E, 128K, 128R, or 128T; 130A, 130D, 130E, 130F, 130H, 130K, 130Q, 130R, 130S, 130T, 130V, 130W, or 130Y; 137D or 137E; 138E or 138L; 145D or 145E; 148F, 148V, or 148W; 149I; 154D, 154K, 154P, or 154V; 156V; 158Y; 160D or 160Q; 166E or 166V; 167L; 168E, 168F, 168R, or 168W; 170F; 192D; 195D or 195E; 199A, 199D, or 199K; 203R or 203V; 206R; 210H; 216T; 219I; 223I, 223L, or 223V; 226R; or any combination thereof.

6. The recombinant carbonic anhydrase polypeptide of claim 5, comprising at least two, three, four, five, six, seven, eight, nine, ten, eleven, twelve, thirteen, fourteen, fifteen, sixteen, seventeen, eighteen, nineteen, or twenty of the residues as defined in claim 5.

7. The recombinant carbonic anhydrase polypeptide of claim 1, comprising the residue(s): 3E; 11P; 18S; 24I; 38D; 35I, 39L, 39H, 39R, 39L, or 39I; 88I; 130S or 130D; 154D or 154P; 223L or 223I; 130S and 154D; 130A and 154D; 130D and 154D; 130A, 154P, and 195E; 15L, 38D, and 128K; 195E and 223I; 25F, 38D, and 128K; 38D and 128K; 38D, 128K and 137E; 38D, 128K, 137E, and 154D; 38D, 128K, 137E, and 154P; 38D, 128K, and 145E; 38D, 128K, and 148W; 38D, 128K, and 160Q; 38D, 128K, and 167L; 38D, 128K, and 168D; 38D, 128K, and 195E; 38D, 128K, and 199A; 38D, 128K, and 216V; 38D, 128K, and 219L; 38D, 128K, and 226K; 38D, E88I, and 128K; 38D, E88K, and 128K; S39I, 128K, 154D, and 223I; S39I, 130A, 154P, 195E, and 223I; 39I, 130A, 154P, 195E, and 223L; 39I, 130A, 154D, and 223L; S39I, 130A, 154D, and 223I; 39I and 195E; 39I and 223I; 39L and 223L; 39L and 223I; or 39I and 223L.

8. The recombinant carbonic anhydrase polypeptide of claim 1, wherein said recombinant carbonic anhydrase polypeptide has increased solubility and/or increased thermostability as compared to said corresponding carbonic anhydrase polypeptide in an alkaline carbonate solution, such as a solution ranging from 1.38 to 1.85 M K₂CO₃ with alpha varying from 0.60 to 0.89.

9. The recombinant carbonic anhydrase polypeptide of claim 1, wherein said recombinant carbonic anhydrase polypeptide has increased solubility as compared to said corresponding carbonic anhydrase polypeptide, after a 24-hour exposure at 22° C. or 70° C. in an alkaline carbonate solution, such as a solution ranging from 1.38 to 1.85 M K₂CO₃ with alpha varying from 0.60 to 0.89.

10. The recombinant carbonic anhydrase polypeptide of claim 1, wherein said recombinant carbonic anhydrase polypeptide has increased solubility as compared to said corresponding carbonic anhydrase polypeptide, after a 24-hour exposure at 80° C. in an alkaline carbonate solution, such as a solution ranging from 1.38 to 1.85 M K₂CO₃ with alpha varying from 0.60 to 0.89.

11. The recombinant carbonic anhydrase polypeptide of claim 1, herein said recombinant carbonic anhydrase polypeptide has increased solubility as compared to said corresponding carbonic anhydrase polypeptide, as determined by titration solubility testing comprising measuring turbidity of 2 g/L of said recombinant carbonic anhydrase polypeptide in solutions ranging from 1.38 to 1.85 M K₂CO₃ with alpha varying from 0.60 to 0.89.

12. The recombinant carbonic anhydrase polypeptide of claim 1 having a solubility of greater than 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12 g/L after 24 hours at 80° C. in an alkaline carbonate solution, such as a solution ranging from 1.38 to 1.85 M K₂CO₃ with alpha varying from 0.60 to 0.89.

13. The recombinant carbonic anhydrase polypeptide of claim 1, wherein said recombinant carbonic anhydrase polypeptide has increased thermostability as compared to said corresponding carbonic anhydrase polypeptide, after a 72-hour exposure at 85° C. in an alkaline carbonate solution, such as a solution ranging from 1.38 to 1.85 M K₂CO₃ with alpha varying from 0.60 to 0.89, or after a 16-hour exposure at 95° C. in an alkaline carbonate solution, such as a solution ranging from 1.38 to 1.85 M K₂CO₃ with alpha varying from 0.60 to 0.89.

14. The recombinant carbonic anhydrase polypeptide of claim 1 comprising an amino acid sequence having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with any one of SEQ ID NOs: 1 to 5.

15. A recombinant carbonic anhydrase polypeptide having carbonic anhydrase activity comprising an amino acid sequence having at least 60% identity with SEQ ID NO: 5, wherein said recombinant carbonic anhydrase polypeptide comprises the residue(s): 3E; 6R; 9A or 9N; 11L, 11P, or 11Y; 15L; 17Y; 18I, 18L, 18R, or 18S; 20K or 20L; 24I, 24M, or 24V; 25F; 27R; 38D, 38R, or 38T; 39H, 35I, 39L, 39R, or 39W; 48L, 48Q, or 48T; 51D, 51E, 51F, 51M, or 51P; 64T; 73E or 73L; 77F; 79E, 79L or 79W; 88E, 88I, 88L, 88R, 88T, or 88V; 105F; 116E or 116R; 119D or 119M; 128E, 128K, 128R, or 128T; 130A, 130D, 130E, 130F, 130H, 130K, 130Q, 130R, 130S, 130T, 130V, 130W, or 130Y; 137D or 137E; 138E or 138L; 145D or 145E; 148F, 148V, or 148W; 149I; 154D, 154K, 154P, or 154V; 156V; 158Y; 160D or 160Q; 166E or 166V; 167L; 168E, 168F, 168R, or 168W; 170F; 192D; 195D or 195E; 199A, 199D, or 199K; 203R or 203V; 206R; 210H; 216T; 219I; 223I, 223L, or 223V; 226R; or any combination thereof.

16. The recombinant carbonic anhydrase polypeptide of claim 15, comprising at least two, three, four, five, six, seven, eight, nine, ten, eleven, twelve, thirteen, fourteen, or fifteen, sixteen, seventeen, eighteen, nineteen, or twenty of the residues as defined in claim 15.

17. The recombinant carbonic anhydrase polypeptide of claim 15, comprising the residue(s): 3E; 11P; 18S; 24I; 38D; 35I, 39L, 39H, 39R, 39L, or 39I; 88I; 130S or 130D; 154D or 154P; 223L or 223I; 130S and 154D; 130A and 154D; 130D and 154D; 130A, 154P, and 195E; 15L, 38D, and 128K; 195E and 223I; 25F, 38D, and 128K; 38D and 128K; 38D, 128K and 137E; 38D, 128K, 137E, and 154D; 38D, 128K, 137E, and 154P; 38D, 128K, and 145E; 38D, 128K, and 148W; 38D, 128K, and 160Q; 38D, 128K, and 167L; 38D, 128K, and 168D; 38D, 128K, and 195E; 38D, 128K, and 199A; 38D, 128K, and 216V; 38D, 128K, and 219L; 38D, 128K, and 226K; 38D, E88I, and 128K; 38D, E88K, and 128K; S39I, 128K, 154D, and 223I; S39I, 130A, 154P, 195E, and 223I; 39I, 130A, 154P, 195E, and 223L; 39I, 130A, 154D, and 223L; S39I, 130A, 154D, and 223I; 39I and 195E; 39I and 223I; 39L and 223L; 39L and 223I; 39I and 223L.

18. The recombinant carbonic anhydrase polypeptide of claim 15, further comprising an amino acid sequence having at least 60% identity with SEQ ID NO: 5 and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223, wherein said recombinant carbonic anhydrase polypeptide has increased solubility and/or increased thermostability as compared to a corresponding carbonic anhydrase polypeptide lacking said one or more amino acid differences.

19. A recombinant carbonic anhydrase polypeptide having carbonic anhydrase activity comprising an amino acid sequence having at least 60% identity with SEQ ID NO: 5, wherein said recombinant carbonic anhydrase polypeptide is engineered to have an isoelectric point (pI) below that of SEQ ID NO: 2, 3 or 4, and has a solubility greater than that of SEQ ID NO: 2, 3 or 4 after 24 hours at 80° C. in an alkaline carbonate solution, such as a solution ranging from 1.38 to 1.85 M K₂CO₃ with alpha varying from 0.60 to 0.89.

20. The recombinant carbonic anhydrase polypeptide of claim 19 having a pI of below 8.3, 8.2. 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, or 6.0.

21. The recombinant carbonic anhydrase polypeptide of claim 19 having a pI of 4 to 8, 5 to 7, 5.5 to 6.5, or 5. to 6.

22. The recombinant carbonic anhydrase polypeptide of claim 19 having a solubility of greater than 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12 g/L after 24 hours at 80° C. in an alkaline carbonate solution, such as a solution ranging from 1.38 to 1.85 M K₂CO₃ with alpha varying from 0.60 to 0.89.

23. The recombinant carbonic anhydrase polypeptide of claim 19, further comprising an amino acid sequence having at least 60% identity with SEQ ID NO: 5 and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223, wherein said recombinant carbonic anhydrase polypeptide has increased solubility and/or increased thermostability as compared to a corresponding carbonic anhydrase polypeptide lacking said one or more amino acid differences.

24. An isolated polynucleotide encoding the recombinant carbonic anhydrase polypeptide as defined in claim 1.

25.-28. (canceled)

29. A method of producing a recombinant carbonic anhydrase polypeptide, the method comprising culturing a host cell comprising the isolated polynucleotide as defined in claim 24 under conditions enabling the expression of the recombinant carbonic anhydrase polypeptide having carbonic anhydrase activity comprising an amino acid sequence having at least 60% identity with SEQ ID NO: 5 and one or more amino acid differences as compared to SEQ ID NO: 1 at residue positions selected from 3, 6, 11, 15, 17, 20, 24, 25, 38, 39, 48, 64, 79, 88, 119, 128, 130, 137, 145, 148, 149, 154, 160, 166, 168, 195, 199, 203, 210, and 223, wherein said recombinant carbonic anhydrase polypeptide has increased solubility and/or increased thermostability as compared to a corresponding carbonic anhydrase polypeptide lacking said one or more amino acid differences, and recovering the recombinant carbonic anhydrase polypeptide.

30. (canceled)

31. Use of the recombinant carbonic anhydrase polypeptide as defined in claim 1 in an industrial process for capturing CO₂ from a CO₂-containing effluent or gas.

32. A process for absorbing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptide as defined in claim 1 to catalyze the hydration reaction of the dissolved CO₂ into bicarbonate and hydrogen ions or the reverse reaction.

33. (canceled)

34. (canceled)

35. The process of claim 32, wherein the absorption solution comprises at least one absorption compound comprising:
(a) a primary amine, a secondary amine, a tertiary amine, a primary alkanolamine, a secondary alkanolamine, a tertiary alkanolamine, a primary amino acid, a secondary amino acid, a tertiary amino acid, dialkylether of polyalkylene glycols, dialkylether or dimethylether of polyethylene glycol, amino acid or a derivative thereof, monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether, or a combination thereof;
(b) a primary amine, a secondary amine, a tertiary amine, a primary alkanolamine, a secondary alkanolamine, a tertiary alkanolamine, a primary amino acid, a secondary amino acid, a tertiary amino acid; or a combination thereof;
(c) dialkylether of polyalkylene glycols, dialkylether or dimethylether of polyethylene glycol, amino acid or derivative thereof, or a combination thereof;
(d) piperazine or derivative thereof, preferably substituted by at least one of alkanol group;
(e) monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether;
(f) an amino acid or derivative thereof, which is preferably a glycine, proline, arginine, histidine, lysine, aspartic acid, glutamic acid, methionine, serine, threonine, glutamine, cysteine, asparagine, valine, leucine, isoleucine, alanine, tyrosine, tryptophan, phenylalanine, taurine, N-cyclohexyl 1,3-propanediamine, N-secondary butyl glycine, N-methyl N-secondary butyl glycine, diethylglycine, dimethylglycine, sarcosine, methyl taurine, methyl-α-aminopropionicacid, N-(β-ethoxy)taurine, N-(β-aminoethyl)taurine, N-methyl alanine, 6-aminohexanoic acid, potassium or sodium salt of the amino acid, or any combination thereof;
(g) a carbonate compound;
(h) sodium carbonate, potassium carbonate, or MDEA;
(i) sodium carbonate; or
(j) potassium carbonate.

36. The process of claim 32, wherein the absorption solution comprises an absorption compound which is a carbonate compound at concentration from about 0.1 to 3 M, 0.5 to 2 M, 1 to 2 M, or 1.25 to 1.75 M. (+

37. The process of claim 36, wherein the carbonate compound is sodium carbonate or potassium carbonate.

38. The process of claim 32, said process further comprising exposing said recombinant carbonic anhydrase polypeptide to a temperature of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C. at some point during said process.

39. The process of claim 32, wherein said CO₂-containing effluent or gas:
(a) comprises between about 0.04 vol % and about 80 vol % of CO₂;
(b) comprises N₂, O₂, noble gases, VOCs, H₂O, CO, SOx, NOx compounds, NH₃, mercaptans, H₂S, H₂, heavy metals, dusts, ashes, or any combination thereof;
(c) is derived from natural gas combustion, coal combustion, biogas combustion, biogas upgrading, or natural gas sweetening; or
(d) any combination of (a) to (c).

40. The process of claim 32, said process further comprising exposing said recombinant carbonic anhydrase polypeptide to a pH from 8 to 11, 8.5 to 11, 9 to 10.5, or 9 to 10 at some point during said process.

41. The process of claim 32, wherein the concentration of the carbonic anhydrase polypeptide in the absorption solution is about 0.01 to 50 g/L, 0.05 to 10 g/L, or 0.1 to 4 g/L.

42. A stock or feed solution comprising the recombinant carbonic anhydrase polypeptide as defined in claim 1 at a concentration of at least 5, 6, 7, 8, 9, 10, 11, or 12 g/L.

43. An isolated polynucleotide encoding the recombinant carbonic anhydrase polypeptide as defined in claim 15.

44. An isolated polynucleotide encoding the recombinant carbonic anhydrase polypeptide as defined in claim 19.

45. A method of producing a recombinant carbonic anhydrase polypeptide, the method comprising culturing a host cell comprising the isolated polynucleotide as defined in claim 43 under conditions enabling the expression of the recombinant carbonic anhydrase polypeptide having carbonic anhydrase activity comprising an amino acid sequence having at least 60% identity with SEQ ID NO: 5, wherein said recombinant carbonic anhydrase polypeptide comprises the residue(s): 3E; 6R; 9A or 9N; 11L, 11P, or 11Y; 15L; 17Y; 18I, 18L, 18R, or 18S; 20K or 20L; 24I, 24M, or 24V; 25F; 27R; 38D, 38R, or 38T; 39H, 35I, 39L, 39R, or 39W; 48L, 48Q, or 48T; 51D, 51E, 51F, 51M, or 51P; 64T; 73E or 73L; 77F; 79E, 79L or 79W; 88E, 88I, 88L, 88R, 88T, or 88V; 105F; 116E or 116R; 119D or 119M; 128E, 128K, 128R, or 128T; 130A, 130D, 130E, 130F, 130H, 130K, 130Q, 130R, 130S, 130T, 130V, 130W, or 130Y; 137D or 137E; 138E or 138L; 145D or 145E; 148F, 148V, or 148W; 149I; 154D, 154K, 154P, or 154V; 156V; 158Y; 160D or 160Q; 166E or 166V; 167L; 168E, 168F, 168R, or 168W; 170F; 192D; 195D or 195E; 199A, 199D, or 199K; 203R or 203V; 206R; 210H; 216T; 219I; 223I, 223L, or 223V; 226R; or any combination thereof, and recovering the recombinant carbonic anhydrase polypeptide.

46. A method of producing a recombinant carbonic anhydrase polypeptide, the method comprising culturing a host cell comprising the isolated polynucleotide as defined in claim 44 under conditions enabling the expression of the recombinant carbonic anhydrase polypeptide having carbonic anhydrase activity comprising an amino acid sequence having at least 60% identity with SEQ ID NO: 5, wherein said recombinant carbonic anhydrase polypeptide is engineered to have an isoelectric point (pI) below that of SEQ ID NO: 2, 3 or 4, and has a solubility greater than that of SEQ ID NO: 2, 3 or 4 after 24 hours at 80° C. in an alkaline carbonate solution, such as a solution ranging from 1.38 to 1.85 M K₂CO₃ with alpha varying from 0.60 to 0.89, and recovering the recombinant carbonic anhydrase polypeptide.

47. Use of the recombinant carbonic anhydrase polypeptide as defined in claim 15 in an industrial process for capturing CO₂ from a CO₂-containing effluent or gas.

48. Use of the recombinant carbonic anhydrase polypeptide as defined in claim 19 in an industrial process for capturing CO₂ from a CO₂-containing effluent or gas.

49. A process for absorbing CO₂ from a co₂-containing effluent or gas, the process comprising: contacting the co₂-containing effluent or gas with an aqueous absorption solution to dissolve the CO₂ into the aqueous absorption solution; and providing the recombinant carbonic anhydrase polypeptide as defined in claim 15 to catalyze the hydration reaction of the dissolved CO₂ into bicarbonate and hydrogen ions or the reverse reaction.

50. A process for absorbing CO₂ from a CO₂-containing effluent or gas, the process comprising: contacting the CO₂-containing effluent or gas with an aqueous absorption solution to dissolve the CO₂ into the aqueous absorption solution; and providing the recombinant carbonic anhydrase polypeptide as defined in claim 19 to catalyze the hydration reaction of the dissolved CO₂ into bicarbonate and hydrogen ions or the reverse reaction.

51. The process of claim 49, wherein the absorption solution comprises at least one absorption compound comprising:
(a) a primary amine, a secondary amine, a tertiary amine, a primary alkanolamine, a secondary alkanolamine, a tertiary alkanolamine, a primary amino acid, a secondary amino acid, a tertiary amino acid, dialkylether of polyalkylene glycols, dialkylether or dimethylether of polyethylene glycol, amino acid or a derivative thereof, monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether, or a combination thereof;
(b) a primary amine, a secondary amine, a tertiary amine, a primary alkanolamine, a secondary alkanolamine, a tertiary alkanolamine, a primary amino acid, a secondary amino acid, a tertiary amino acid; or a combination thereof;
(c) dialkylether of polyalkylene glycols, dialkylether or dimethylether of polyethylene glycol, amino acid or derivative thereof, or a combination thereof;
(d) piperazine or derivative thereof, preferably substituted by at least one of alkanol group;
(e) monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether;
(f) an amino acid or derivative thereof, which is preferably a glycine, proline, arginine, histidine, lysine, aspartic acid, glutamic acid, methionine, serine, threonine, glutamine, cysteine, asparagine, valine, leucine, isoleucine, alanine, tyrosine, tryptophan, phenylalanine, taurine, N-cyclohexyl 1,3-propanediamine, N-secondary butyl glycine, N-methyl N-secondary butyl glycine, diethylglycine, dimethylglycine, sarcosine, methyl taurine, methyl-α-aminopropionicacid, N-(β-ethoxy)taurine, N-(β-aminoethyl)taurine, N-methyl alanine, 6-aminohexanoic acid, potassium or sodium salt of the amino acid, or any combination thereof;
(g) a carbonate compound;
(h) sodium carbonate, potassium carbonate, or MDEA;
(i) sodium carbonate; or
(j) potassium carbonate.

52. The process of claim 49, wherein the absorption solution comprises an absorption compound which is a carbonate compound at concentration from about 0.1 to 3 M, 0.5 to 2 M, 1 to 2 M, or 1.25 to 1.75 M.

53. The process of claim 52, wherein the carbonate compound is sodium carbonate or potassium carbonate.

54. The process of claim 49, said process further comprising exposing said recombinant carbonic anhydrase polypeptide to a temperature of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C. at some point during said process.

55. The process of claim 49, wherein said CO₂-containing effluent or gas:
(a) comprises between about 0.04 vol % and about 80 vol % of CO₂;
(b) comprises N₂, O₂, noble gases, VOCs, H₂O, CO, SOx, NOx compounds, NH₃, mercaptans, H₂S, H₂, heavy metals, dusts, ashes, or any combination thereof;
(c) is derived from natural gas combustion, coal combustion, biogas combustion, biogas upgrading, or natural gas sweetening; or
(d) any combination of (a) to (c).

56. The process of claim 49, said process further comprising exposing said recombinant carbonic anhydrase polypeptide to a pH from 8 to 11, 8.5 to 11, 9 to 10.5, or 9 to 10 at some point during said process.

57. The process of claim 49, wherein the concentration of the carbonic anhydrase polypeptide in the absorption solution is about 0.01 to 50 g/L, 0.05 to 10 g/L, or 0.1 to 4 g/L.

58. A stock or feed solution comprising the recombinant carbonic anhydrase polypeptide as defined in claim 15 at a concentration of at least 5, 6, 7, 8, 9, 10, 11, or 12 g/L.

59. The process of claim 50, wherein the absorption solution comprises at least one absorption compound comprising:
(a) a primary amine, a secondary amine, a tertiary amine, a primary alkanolamine, a secondary alkanolamine, a tertiary alkanolamine, a primary amino acid, a secondary amino acid, a tertiary amino acid, dialkylether of polyalkylene glycols, dialkylether or dimethylether of polyethylene glycol, amino acid or a derivative thereof, monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether, or a combination thereof;
(b) a primary amine, a secondary amine, a tertiary amine, a primary alkanolamine, a secondary alkanolamine, a tertiary alkanolamine, a primary amino acid, a secondary amino acid, a tertiary amino acid; or a combination thereof;
(c) dialkylether of polyalkylene glycols, dialkylether or dimethylether of polyethylene glycol, amino acid or derivative thereof, or a combination thereof;
(d) piperazine or derivative thereof, preferably substituted by at least one of alkanol group;
(e) monoethanolamine (MEA), 2-amino-2-methyl-1-propanol (AMP), 2-(2-aminoethylamino)ethanol (AEE), 2-amino-2-hydroxymethyl-1,3-propanediol (Tris or AHPD), N-methyldiethanolamine (MDEA), dimethylmonoethanolamine (DMMEA), diethylmonoethanolamine (DEMEA), triisopropanolamine (TIPA), triethanolamine (TEA), diethanolamine (DEA), diisopropylamine (DIPA), methylmonoethanolamine (MMEA), tertiarybutylaminoethoxy ethanol (TBEE), N-2-hydroxyethyl-piperzine (HEP), 2-amino-2-hydroxymethyl-1,3-propanediol (AHPD), hindered diamine (HDA), bis-(tertiarybutylaminoethoxy)-ethane (BTEE), ethoxyethoxyethanol-tertiarybutylamine (EEETB), bis-(tertiarybutylaminoethyl)ether, 1,2-bis-(tertiarybutylaminoethoxy)ethane and/or bis-(2-isopropylaminopropyl)ether;
(f) an amino acid or derivative thereof, which is preferably a glycine, proline, arginine, histidine, lysine, aspartic acid, glutamic acid, methionine, serine, threonine, glutamine, cysteine, asparagine, valine, leucine, isoleucine, alanine, tyrosine, tryptophan, phenylalanine, taurine, N-cyclohexyl 1,3-propanediamine, N-secondary butyl glycine, N-methyl N-secondary butyl glycine, diethylglycine, dimethylglycine, sarcosine, methyl taurine, methyl-α-aminopropionicacid, N-(β-ethoxy)taurine, N-(β-aminoethyl)taurine, N-methyl alanine, 6-aminohexanoic acid, potassium or sodium salt of the amino acid, or any combination thereof;
(g) a carbonate compound;
(h) sodium carbonate, potassium carbonate, or MDEA;
(i) sodium carbonate; or
(j) potassium carbonate.

60. The process of claim 50, wherein the absorption solution comprises an absorption compound which is a carbonate compound at concentration from about 0.1 to 3 M, 0.5 to 2 M, 1 to 2 M, or 1.25 to 1.75 M.

61. The process of claim 60, wherein the carbonate compound is sodium carbonate or potassium carbonate.

62. The process of claim 50, said process further comprising exposing said recombinant carbonic anhydrase polypeptide to a temperature of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C. at some point during said process.

63. The process of claim 50, wherein said CO₂-containing effluent or gas:
(a) comprises between about 0.04 vol % and about 80 vol % of CO₂;
(b) comprises N₂, O₂, noble gases, VOCs, H₂O, CO, SOx, NOx compounds, NH₃, mercaptans, H₂S, H₂, heavy metals, dusts, ashes, or any combination thereof;
(c) is derived from natural gas combustion, coal combustion, biogas combustion, biogas upgrading, or natural gas sweetening; or
(d) any combination of (a) to (c).

64. The process of claim 50, said process further comprising exposing said recombinant carbonic anhydrase polypeptide to a pH from 8 to 11, 8.5 to 11, 9 to 10.5, or 9 to 10 at some point during said process.

65. The process of claim 50, wherein the concentration of the carbonic anhydrase polypeptide in the absorption solution is about 0.01 to 50 g/L, 0.05 to 10 g/L, or 0.1 to 4 g/L.

66. A stock or feed solution comprising the recombinant carbonic anhydrase polypeptide as defined in claim 19 at a concentration of at least 5, 6, 7, 8, 9, 10, 11, or 12 g/L.


SEQUENCE LISTING (20 sequences)

SEQ ID NO: 1 - protein, 226 aa, Artificial Sequence
  Wild-type TACA sequence with N terminus modified for improved bacterial expression
MEHEWGYSGSIGPEHWGDLSPEYLMCKIGKNQSPIDINSADAVKACLAPVSVYYVSDAKYVVNNGHTIKVVMGGRGYVVVDGKRFYLKQFHFHAPSEHTVNGKHYPFEAHFVHLDKNGNITVLGVFFKVGKENPELEKVWRVMPEEPGQKRHLTARIDPEKLLPENRDYYRYSGSLTTPPCSEGVRWIVFKEPVEMSREQLEKFRKVMGFDNNRPVQPLNARKVMK

SEQ ID NO: 2 - protein, 226 aa, Artificial Sequence
  SEQ ID NO 1 + R156E
MEHEWGYSGSIGPEHWGDLSPEYLMCKIGKNQSPIDINSADAVKACLAPVSVYYVSDAKYVVNNGHTIKVVMGGRGYVVVDGKRFYLKQFHFHAPSEHTVNGKHYPFEAHFVHLDKNGNITVLGVFFKVGKENPELEKVWRVMPEEPGQKRHLTAEIDPEKLLPENRDYYRYSGSLTTPPCSEGVRWIVFKEPVEMSREQLEKFRKVMGFDNNRPVQPLNARKVMK

SEQ ID NO: 3 - protein, 226 aa, Artificial Sequence
  SEQ ID NO 1 + K27R, N38D, K88R, K116R, N119D, K128R, R156E, E160D, D168E, E192D, E199D, K203R, K206R, V216T, L219I
MEHEWGYSGSIGPEHWGDLSPEYLMCRIGKNQSPIDIDSADAVKACLAPVSVYYVSDAKYVVNNGHTIKVVMGGRGYVVVDGKRFYLRQFHFHAPSEHTVNGKHYPFEAHFVHLDRNGDITVLGVFFRVGKENPELEKVWRVMPEEPGQKRHLTAEIDPDKLLPENREYYRYSGSLTTPPCSEGVRWIVFKDPVEMSRDQLERFRRVMGFDNNRPTQPINARKVMK

SEQ ID NO: 4 - protein, 226 aa, Artificial Sequence
  SEQ ID NO 1 + Y77F, V79E, K88E, Y105F, K116E, K128E, E137D, E145D, R156E, D168E, Y170F, E195D, E199D, V216T, L219I, K226R
MEHEWGYSGSIGPEHWGDLSPEYLMCKIGKNQSPIDINSADAVKACLAPVSVYYVSDAKYVVNNGHTIKVVMGGRGFVEVDGKRFYLEQFHFHAPSEHTVNGKHFPFEAHFVHLDENGNITVLGVFFEVGKENPELDKVWRVMPDEPGQKRHLTAEIDPEKLLPENREYFRYSGSLTTPPCSEGVRWIVFKEPVDMSRDQLEKFRKVMGFDNNRPTQPINARKVMR

SEQ ID NO: 5 - protein, 226 aa, Artificial Sequence
  Amalgam of SEQ ID NOs 2-4
  VARIANT (27)..(27): Xaa can be K or R
  VARIANT (38)..(38): Xaa can be N or D
  VARIANT (77)..(77): Xaa can be Y or F
  VARIANT (79)..(79): Xaa can be V or E
  VARIANT (88)..(88): Xaa can be K, R, or E
  VARIANT (105)..(105): Xaa can be Y or F
  VARIANT (116)..(116): Xaa can be K, R, or E
  VARIANT (119)..(119): Xaa can be N or D
  VARIANT (128)..(128): Xaa can be K, R, or E
  VARIANT (137)..(137): Xaa can be E or D
  VARIANT (145)..(145): Xaa can be E or D
  VARIANT (156)..(156): Xaa can be R or E
  misc_feature (160)..(160): Xaa can be any naturally occurring amino acid
  VARIANT (168)..(168): Xaa can be D or E
  VARIANT (170)..(170): Xaa can be Y or F
  VARIANT (192)..(192): Xaa can be E or D
  VARIANT (195)..(195): Xaa can be E or D
  VARIANT (199)..(199): Xaa can be E or D
  VARIANT (203)..(203): Xaa can be K or R
  VARIANT (206)..(206): Xaa can be K or R
  VARIANT (216)..(216): Xaa can be V or T
  VARIANT (219)..(219): Xaa can be L or I
  VARIANT (226)..(226): Xaa can be K or R
MEHEWGYSGSIGPEHWGDLSPEYLMCXIGKNQSPIDIXSADAVKACLAPVSVYYVSDAKYVVNNGHTIKVVMGGRGXVXVDGKRFYLXQFHFHAPSEHTVNGKHXPFEAHFVHLDXNGXITVLGVFFXVGKENPELXKVWRVMPXEPGQKRHLTAXIDPXKLLPENRXYXRYSGSLTTPPCSEGVRWIVFKXPVXMSRXQLEXFRXVMGFDNNRPXQPXNARKVMX

SEQ ID NO: 6 - protein, 226 aa, Artificial Sequence
  SEQ ID NO 4 + S39I, E128K, T154D, K223I
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFKVGKENPELDKVWRVMPDEPGQKRHLDAEIDPEKLLPENREYFRYSGSLTTPPCSEGVRWIVFKEPVEMSRDQLEKFRKVMGFDNNRPTQPINARIVMR

SEQ ID NO: 7 - protein, 226 aa, Artificial Sequence
  SEQ ID NO 4 + S39I, G130A, T154D, K223I
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFEVAKENPELDKVWRVMPDEPGQKRHLDAEIDPEKLLPENREYFRYSGSLTTPPCSEGVRWIVFKEPVEMSRDQLEKFRKVMGFDNNRPTQPINARIVMR

SEQ ID NO: 8 - protein, 226 aa, Artificial Sequence
  SEQ ID NO 4 + S39I, G130A, T154D, K223L
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFEVAKENPELDKVWRVMPDEPGQKRHLDAEIDPEKLLPENREYFRYSGSLTTPPCSEGVRWIVFKEPVEMSRDQLEKFRKVMGFDNNRPTQPINARLVMR

SEQ ID NO: 9 - protein, 226 aa, Artificial Sequence
  SEQ ID NO 4 + S39I, G130A, T154P, D195E, K223I
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFEVAKENPELDKVWRVMPDEPGQKRHLPAEIDPEKLLPENREYFRYSGSLTTPPCSEGVRWIVFKEPVEMSRDQLEKFRKVMGFDNNRPTQPINARIVMR

SEQ ID NO: 10 - protein, 226 aa, Artificial Sequence
  SEQ ID NO 4 + S39I, G130A, T154P, D195E, K223L
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFEVAKENPELDKVWRVMPDEPGQKRHLPAEIDPEKLLPENREYFRYSGSLTTPPCSEGVRWIVFKEPVEMSRDQLEKFRKVMGFDNNRPTQPINARLVMR

SEQ ID NO: 11 - protein, 226 aa, Artificial Sequence
  SEQ ID NO 4 + S39I, K223L
MEHEWGYSGSIGPEHWGDLSPEYLMCKIGKNQSPIDINIADAVKACLAPVSVYYVSDAKYVVNNGHTIKVVMGGRGFVEVDGKRFYLEQFHFHAPSEHTVNGKHFPFEAHFVHLDENGNITVLGVFFEVGKENPELDKVWRVMPDEPGQKRHLTAEIDPEKLLPENREYFRYSGSLTTPPCSEGVRWIVFKEPVDMSRDQLEKFRKVMGFDNNRPTQPINARLVMR

SEQ ID NO: 12 - protein, 243 aa, Persephonella marina (WP_015898908.1)
MKKFVVGLSSLVLATSSFAGGGWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSEGVRWIVMEEEMEMSKEQIEKFRKIMGGDTNRPVQPLNARMIMEK

SEQ ID NO: 13 - protein, 246 aa, Persephonella sp. KM09-Lau-8 (WP_029522463.1)
MRKTTFVAGLSFLIMGYGAYSGEHWSYHGKTGPEHWGDLKPEFIMCKIGKNQSPVDINRIVEAELKELKISYRPGASYLVNNGHTIKVSYGKGSYLEVDGTKFELKQFHFHAPSEHKIKGKLYPFEAHFVHMDKNGNIAVIGVLFKEGRKNPVIEKLWKNISSKVGEKVKITDKINAYDLLPKKKRYYRYSGSLTTPPCSEGVRWFVMVEEMELSKEQIEKFRKLMGGDTNRPVQPLNARMVMKQD

SEQ ID NO: 14 - protein, 247 aa, Persephonella sp. IF05-L8 (WP_029521561.1)
MKKKLLSVSIASAVFSFAFAGGNGDWSYHGSTGPEHWGDLKEEYIMCKIGKNQSPVDINRIVEAELEEIKISYKPGASSLVNNGHTIKVSYEGGSYIVIDGIKFELKQFHFHAPSEHKIKGKLYPFEAHFVHADKDGNLAVIGVVFKEGKKNPVIEKLWKNLPSKVGEEVKLSEKINAYDLLPKKKKYFRYSGSLTTPPCSEGVRWIVMTKEMELSKEQIEKFRKLMGGDTNRPVQPLNARMIMIED

SEQ ID NO: 15 - protein, 244 aa, uncultured bacterium (AVN84966.1)
MKKLAFIIFSLTIGTVIAGGVGHWSYHGETGPQHWGDLKNEYIMCKIGKNQSPVDISRIVEAELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYSGSLTTPPCSEGVRWIVMEEEMELSKEQIEKFRKLMGGDTNRPVQPLNARMIMEMD

SEQ ID NO: 16 - protein, 247 aa, Persephonella hydrogeniphila (WP_096999253.1)
MKKAVLSVATASAIFGIAVAGGAGSWSYHGATGPEHWGDLKDEYIMCKIGKNQSPVDISRIAEADLEDIKFHYSPGATSLLNNGHTIKVSYEPGSYIVVDGIRFELKQFHFHAPSEHTVKGKSYPFEAHFVHADKDGNLAVIGVLFKEGKENPVIKKLWENLPSHTGEKVKLAHKINAYDLLPKRKKYYRYSGSLTTPPCSEGVRWLVMTTEMEMSKEQIEKFRKLMGGDTNRPVQPVNARMILIKD

SEQ ID NO: 17 - protein, 245 aa, Aquificae bacterium (RMD45622.1)
MRKKVLGLILALSFSNYSIAGEWSYHGEHGPEHWGDIKKEFIMCKIGKNQSPVDLNRIVKAELKEVDINYKPGGISALNNGHTIKVSYEPGSYIVIDGIKFELKQFHFHSPSEHKIKGKQYPLEAHFVHADKDGNLAVIGVVFKEGKENPIIKQIWENLPMEEGKEVKLSQKVNAYELLPEDKNYYRYSGSLTTPPCSEGVRWIVMEKPLEVSKEQIEKFRKAMGNVDTNRPVQPLNARLILEKE

SEQ ID NO: 18 - protein, 249 aa, Caminibacter mediatlanticus (WP_007474387.1)
MKKFLLGLGVAAITMLSASSYNYHATWSYSGKTGPEYWGDLKKEYQMCKIGKNQSPIDIKTNSTQTFNTNLKPFKIKYLGKGYEVINNGHTIKVKTEGKNCVRIDGIKFKLAQLHFHTPSENTINGKHFPMEAHYVHLDKNGNITVLAVMYKIGKENKSLNKMLAVLPTKVGEENKVMGNLNPMELLPKNKAYYRFNGSLTTPPCSEGVRWIVFKTPVEISQAQYEKMHAVMGTNNRPVQPINARVILK

SEQ ID NO: 19 - protein, 248 aa, Hydrogenimonas sp. (BBG65557.1)
MKKITSLFLGAATASVLMAGGHGVEWGYTGHTGPEHWGDLSDKYIMCKLGKNQSPIDIKTADAFETDLPEIAIDYKSASVNEVNNGHTVQVNIVPGSSITVDGIKFELKQFHFHTPSENTIDGRYFPLEAHFVHADENGNLAVIGVMFEYGKENPVLKSVWERMPEHAKDVEKITLSAEEVKNLLPANKEYYRFNGSLTTPPCSEGVRWFVMKEPVTVSKDQVKKFAEVMHHDNNRPVQPVNARIILK

SEQ ID NO: 20 - protein, 249 aa, Hydrogenimonas sp. (RUM45284.1)
MKKVVSLLIGSAVAATLVMAQEHTIHWGYSGHGGPEHWGELSKDYQMCLIGKNQSPIDIRTKSAFETKLPAIQFDYHTSAKDVVDNGHTEQVDLVPGSSITIDGRKFELKQFHFHTPSENRIDGKSFPLEAHFVHADEHGNLAVVALMFTYGEANPVLAKVWENMPEHAGEKHHFTLSSEDVMKLLPKNRDYYRFNGSLTTPPCTEGVRWFVIKEPVTISKEQVQKFAKIMHHNNNRPVQPLNSRMILK